(19) United States
(12) Patent Application Publication
(10) Pub. No.: US 2021/0292381 A1
(43) Pub. Date: Sep. 23, 2021

(54) METHODS OF DE-EPITOPING WHEAT PROTEINS AND USE OF SAME FOR THE TREATMENT OF CELIAC DISEASE

(71) Applicant: Ukko Inc., San Francisco, CA (US)

(72) Inventors: Yanay OFRAN, Tel-Aviv (IL); Moshe BEN-DAVID, Rehovot (IL); Assaf BIRAN, Tel-Aviv (IL); Shiri ZAKIN, Sde Warburg (IL); Orly MARCU, ModiIn (IL)

(73) Assignee: Ukko Inc., San Francisco, CA (US)

(21) Appl. No.: 17/257,604

(22) PCT Filed: Jul. 4, 2019
(86) PCT No.: PCT/IB2019/055724

§ 371 Date: Jan. 4, 2021

Related U.S. Application Data
(60) Provisional application No. 62/693,925, filed on Jul. 4, 2018.

(51) Int. Cl. C07K 14/415; G16B 40/00; C12N 15/82; A21D 2/26; A21D 13/066; A23J 3/18

(52) U.S. Cl. CPC C07K 14/415; G16B 40/00; A23J 3/18; A21D 2/265; A21D 13/066; C12N 15/8257

(57) ABSTRACT

A method for identifying an epitope of a wheat T cell immunogen is provided. The method comprises identifying an epitope on the wheat T cell immunogen for the ability to bind a major histocompatibility complex (MHC) class II, thereby identifying the epitope of a wheat T cell immunogen.

### RELATED APPLICATION

This application claims the benefit of priority from U.S. Provisional Patent Application No. 62/693,925 filed on 4 Jul. 2018, the contents of which are incorporated herein by reference in their entirety.

### FIELD AND BACKGROUND OF THE INVENTION

The present invention, in some embodiments thereof, relates to methods of de-epitoping wheat proteins and use of same for the treatment of gluten sensitivity, including celiac disease.

Celiac disease (CD) is an acquired chronic immune disorder that develops in susceptible individuals (many of whom are of HLA genotype DQ2 or DQ8) related to an environmental factor, gluten, which is the storage protein of wheat and related grains like rye and barley. The prevalence of celiac disease in Europe and in the United States has been estimated to be approximately 1-2% of the population. Celiac disease has a wide range of clinical manifestations including latent or silent celiac disease, disease with only mild gastrointestinal disturbances, chronic gastrointestinal symptoms, malabsorption, and/or weight loss. Celiac disease is often diagnosed in patients with isolated iron deficiency anemia.

The ingestion of gluten-containing cereals can also induce manifestations outside the gut, such as osteoporosis, peripheral and central nervous system involvement, mild or severe liver disease, infertility problems, and the classical example is the gluten-induced skin disease, dermatitis herpetiformis.

For patients with celiac disease, lifelong complete gluten exclusion needs to be strictly followed to avoid a substantially enhanced risk for the development of further complications, such as bone disorders, infertility, and cancer. The mortality rate in patients with celiac disease exceeds that of the general population; however, there is a trend towards reduction in mortality after 1-5 years on a gluten-free diet.

Following a completely gluten-free diet is, however, very challenging. Even highly motivated patients who try to maintain a strict dietary regimen are affected due to inadvertent or background exposure to gluten. As many as 80% of patients with celiac disease who are in clinical remission and who claim to be following a gluten-free diet, have persistent abnormalities in small bowel biopsy specimens. Inadvertent exposure to gluten has been identified as the leading cause of non-responsive celiac disease among clinically diagnosed patients who were presumed to be on a gluten-free diet.

Taken together, there is an acute need for additional dietary therapies for celiac disease which are both non-costly and accessible.

Sanchez-Leon, Susana et al. “Low-gluten, Nontransgenic Wheat Engineered with CRISPR/Cas9.” Plant Biotechnology Journal 16.4 (2018): 902-910. PMC.

Additional background art includes US Patent Application No. 20160338366.

### SUMMARY OF THE INVENTION

According to an aspect of some embodiments of the present invention there is provided a method for identifying an epitope of a wheat T cell immunogen, the method comprising identifying an epitope on the wheat T cell immunogen for the ability to bind a major histocompatibility complex (MHC) class II, thereby identifying the epitope of a wheat T cell immunogen.

According to an aspect of some embodiments of the present invention there is provided a method for de-epitoping a wheat polypeptide, the method comprising mutating one or more amino acid residues of a celiac-associated epitope on the wheat polypeptide to generate a de-epitoped polypeptide having one or more mutation in the celiac-associated epitope, thereby de-epitoping the wheat polypeptide.

According to some embodiments of the invention, the epitope is a celiac-associated epitope.

According to some embodiments of the invention, the mutating one or more amino acids does not reduce the allergenicity of the wheat polypeptide.

According to some embodiments of the invention, the wheat polypeptide is a glutenin or a gliadin.

According to some embodiments of the invention, the gliadin is selected from the group consisting of α/β-gliadin, γ-gliadin and ω-gliadin.

According to some embodiments of the invention, the method further comprises computationally predicting an epitope on the wheat polypeptide for the ability to bind a major histocompatibility complex (MHC) class II wherein the predicting is performed prior to the mutating.

According to some embodiments of the invention, the MHC II is HLA-DQ2 or HLA-DQ8.

According to some embodiments of the invention, the method further comprises computationally predicting an epitope on the wheat polypeptide from the amino acid sequence of antigen binding regions of T cell receptors (TCRs) which bind to the wheat polypeptide.

According to some embodiments of the invention, the method comprises validating the identified epitope experimentally.

According to some embodiments of the invention, the identifying is by computationally predicting the epitope and is optionally performed using a machine-learning algorithm trained to recognize residue pairing preferences on a dataset of MHC II-peptide complexes.

According to some embodiments of the invention, said identifying is by computationally predicting the epitope and is optionally performed using a machine-learning algorithm trained to recognize whether a given MHC II and a given T-cell immunogen are likely to bind each other based on a data set of MHC II-peptide interactions.

According to some embodiments of the invention, computationally predicting the epitope is performed using an algorithm trained to predict TCR-peptide interactions for peptides derived from the wheat polypeptide.

According to some embodiments of the invention, validating the computationally predicted epitope experimentally comprises performing a MHCII binding assay.

According to some embodiments of the invention, the mutating comprises mutating at least two amino acid residues of the identified epitope.

According to some embodiments of the invention, the mutation is a conservative mutation.

According to some embodiments of the invention, the mutation is a non-conservative mutation.

According to some embodiments of the invention, the de-epitoped polypeptide binds with a lower affinity to T-cells derived from a celiac patient than a corresponding non-mutated polypeptide binds to T cells derived from the celiac patient.

According to some embodiments of the invention, the de-epitoped polypeptide activates T-cells derived from a celiac patient to a lesser extent that a corresponding non-mutated polypeptide activates T cells derived from the celiac patient.

According to some embodiments of the invention, the affinity is reduced by at least about 10%.

According to some embodiments of the invention, the mutation does not disrupt the function of the polypeptide.

According to some embodiments of the invention, the mutation does not disrupt the three-dimensional structure of the polypeptide.

According to some embodiments of the invention, the mutation does not disrupt folding of the polypeptide.

According to an aspect of some embodiments of the present invention there is provided an isolated glutenin or gliadin polypeptide being mutated compared to the corresponding wild-type glutenin or gliadin polypeptide such that it binds with a lower affinity to T-cells derived from a celiac patient than a corresponding non-mutated polypeptide binds to T cells derived from the celiac patient.

According to an aspect of some embodiments of the present invention there is provided an isolated polynucleotide encoding the isolated glutenin or gliadin polypeptide.

According to an aspect of some embodiments of the present invention there is provided an expression vector comprising the isolated polynucleotide, operatively linked to a transcriptional regulatory sequence so as to allow expression of the glutenin or gliadin in a plant cell.

According to some embodiments of the invention, the transcriptional regulatory sequence comprises a plant promoter.

According to some embodiments of the invention, the plant promoter comprises a wheat promoter.

According to an aspect of some embodiments of the present invention there is provided a flour derived from a gluten-free plant, comprising a de-epitoped glutenin or gliadin polypeptide.

According to some embodiments of the invention, the de-epitoped glutenin or gliadin polypeptide is the isolated glutenin or gliadin polypeptide of claim 24.

According to an aspect of some embodiments of the present invention there is provided a dough comprising the flour.

According to some embodiments of the invention, the dough is characterized by at least one property selected from the group consisting of: a higher development time (DT), a lower stability time (S), a higher degree of softening (DS), a higher consistency (C) value and any combination thereof, as compared to a corresponding dough being absent of the de-epitoped glutenin or gliadin polypeptide.

According to some embodiments of the invention, the dough is characterized by at least one property selected from the group consisting of: a. higher rigidity relative to a corresponding dough being absent of the de-epitoped glutenin or gliadin polypeptide; b. higher stability to mechanical solicitations relative to a corresponding dough being absent of the de-epitoped glutenin or gliadin polypeptide; c. higher critical tension value relative to a corresponding dough being absent of the de-epitoped glutenin or gliadin polypeptide; d. a lower deformation capacity relative to a corresponding dough being absent of the de-epitoped glutenin or gliadin polypeptide; e. has higher plasticity relative to a corresponding dough being absent of the de-epitoped glutenin or gliadin polypeptide; and f. higher consistency relative to a corresponding dough being absent of the de-epitoped glutenin or gliadin polypeptide.

According to some embodiments of the invention, the dough additionally comprises salt.

According to some embodiments of the invention, the dough is combined with at least one additional food ingredient, the at least one additional food ingredient is selected from the group consisting of flavoring agent, vegetable or vegetable part, oil, vitamins and olives.

According to some embodiments of the invention, the dough further comprises a leavening agent, the leavening agent is selected from the group consisting of: unpasteurized beer, buttermilk, ginger beer, kefir, sourdough starter, yeast, whey protein concentrate, yogurt, biological leaveners, chemical leaveners, baking soda, baking powder, baker's ammonia, potassium bicarbonate and any combination thereof.

According to an aspect of some embodiments of the present invention there is provided a wheat being genetically modified to express the isolated glutenin or gliadin polypeptide.

According to some embodiments of the invention, expression of the corresponding non-mutated polypeptide is down-regulated compared to a wild-type wheat.

According to some embodiments of the invention, the wheat comprises an RNA silencing agent directed towards the non-mutated polypeptide.

According to some embodiments of the invention, the wheat is genetically modified by a DNA editing agent.

According to an aspect of some embodiments of the present invention there is provided a flour generated from the wheat.

According to an aspect of some embodiments of the present invention there is provided a dough generated from the wheat.

According to an aspect of some embodiments of the present invention there is provided a processed dough product prepared by processing the dough, the processing being selected from the group consisting of combining the dough with a food ingredient, rising, kneading, extruding, molding, shaping, cooking, stewing, boiling, broiling, baking, frying and any combination of same.

According to some embodiments of the invention, the processed dough product is in a form selected from the group consisting of a pan bread, a pizza bread crust, a pasta, a tortilla, a Panini bread, a pretzel, a pie and a sandwich bread product.

According to an aspect of some embodiments of the present invention there is provided a method of producing flour comprising processing the wheat, thereby producing the flour.

According to some embodiments of the invention, the processing comprises grinding or milling.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 illustrates a library design strategy according to embodiments of the present invention.

FIGS. 2A-C are photographs of the bread baking process (FIG. 2A), dough (FIG. 2B) and baked bread (FIG. 2C) with isolated gluten and non-wheat flour.

### DESCRIPTION OF SPECIFIC EMBODIMENTS OF THE INVENTION

The present invention, in some embodiments thereof, relates to methods of de-epitoping wheat proteins and use of same for the treatment of celiac disease.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The present disclosure provides methods for identifying one or more epitopes on wheat polypeptides which are responsible for eliciting an autoimmune response in a subject with celiac disease, as well as methods for de-epitoping these wheat polypeptides. Advantageously, the methods described herein provide, for the first time, a combination of methods for identifying the epitopes on wheat polypeptides, and methods of de-epitoping these polypeptides, thus providing polypeptides with reduced or eliminated ability to elicit an immune response. Without wishing to be bound by theory, the methods described herein are believed to provide a number of beneficial outcomes for celiac patients, their families, and society at large: 1) providing foods and products that are safe to handle and consume for celiac patients, 2) allowing celiac patients to no longer avoid consuming otherwise healthy foods (thereby addressing the nutritional disadvantages of food avoidance), 3) increasing use of non-harmful foods, 4) reducing the negative economic impacts of celiac disease, 5) reducing/eliminating the need to perform potentially risky treatments on celiac patients, 6) mapping epitopes for individual celiac patients, thus offering the possibility of personalized tests that assess the risks of each person for various foods, and 7) mapping epitopes for individual celiac patients, thus offering the possibility of personalized nutrition recommendations.

Thus, according to a first aspect of the present invention, there is provided a method for identifying an epitope of a wheat T cell immunogen, the method comprising identifying (e.g., computationally predicting) an epitope on the wheat T cell immunogen for the ability to bind a major histocompatibility complex (MHC) class II, thereby identifying the epitope of a wheat T cell immunogen.

As used herein, the term “epitope” refers to a determinant that is recognized by lymphocytes. The epitope can be a peptide which is presented by a major histocompatibility complex (MHC) molecule and is capable of specifically binding to a T-cell receptor. In certain embodiments, an epitope is a region of a T cell immunogen that is specifically bound by a T-cell receptor. In certain embodiments, an epitope may include chemically active surface groupings of molecules such as amino acids, sugar side chains, phosphoryl, or sulfonyl groups. In certain embodiments, an epitope may have specific three-dimensional structural characteristics and/or specific charge characteristics.

The T cell epitope of this aspect of the present invention is typically a short peptide that is bound to a class I or II MHC molecule thus forming a ternary complex that can be recognized by a T-cell bearing a matching T-cell receptor binding to the MHC/peptide complex with appropriate affinity. Peptides binding to MHC class I molecules are typically about 8-14 amino acids in length but can be longer. T-cell epitopes that bind to MHC class II molecules are typically about 12-30 amino acids in length, but can be longer. In the case of peptides that bind to MHC class II molecules, the same peptide and corresponding T cell epitope may share a common core segment, but differ in the overall length due to flanking sequences of differing lengths upstream of the amino-terminus of the core sequence and downstream of its carboxy terminus, respectively. A T-cell epitope may be classified as an antigen if it elicits an immune response.

The molecules that transport and present peptides on the cell surface are referred to as proteins of the major histocompatibility complex (MHC). MHC proteins are classified into two types, referred to as MHC class I and MHC class II. The structures of the proteins of the two MHC classes are very similar; however, they have very different functions. Proteins of MHC class I are present on the surface of almost all cells of the body, including most tumor cells. MHC class I proteins are loaded with antigens that usually originate from endogenous proteins or from pathogens present inside cells, and are then presented to naive or cytotoxic T-lymphocytes (CTLs). MHC class II proteins are present on dendritic cells, B-lymphocytes, macrophages and other antigen-presenting cells. They mainly present peptides, which are processed from external antigen sources, i.e. outside of the cells, to T-helper (Th) cells. T-Cell receptors are capable of recognizing and binding peptides complexed with the molecules of MHC class I or II. Each cytotoxic T-lymphocyte expresses a specific T-cell receptor which is capable of binding specific MHC/peptide complexes.

Antigen presenting cells (APC) are cells which present peptide fragments of protein antigens in association with MHC molecules on their cell surface. Some APCs may activate antigen specific T cells. Examples of APCs include, but are not limited to dendritic cells, beta cells and macrophages.

According to a particular embodiment, the T cell epitope is a celiac disease-associated epitope—i.e. the epitope is presented on antigen presenting cells (APCs) of a Celiac patient.

The present teachings also relate to other forms of gluten sensitivity. The term celiac disease is meant to encompass those forms in certain embodiments.

Celiac disease is a long-term autoimmune disorder that primarily affects the small intestine. Classic symptoms include gastrointestinal problems such as chronic diarrhea, abdominal distention, malabsorption, loss of appetite and among children failure to grow normally. This often begins between six months and two years of age. Non-classic symptoms are more common, especially in people older than two years. There may be mild or absent gastrointestinal symptoms, a wide number of symptoms involving any part of the body or no obvious symptoms.

Celiac disease is caused by a reaction to gluten, which are various proteins found in wheat and in other grains such as barley and rye. Upon exposure to gluten, an abnormal immune response may lead to the production of several different autoantibodies that can affect a number of different organs. In the small bowel, this causes an inflammatory reaction and may produce shortening of the villi lining the small intestine.

Diagnosis is typically made by a combination of blood antibody tests and intestinal biopsies, helped by specific genetic testing. While the disease is caused by a permanent intolerance to wheat proteins, it is not a form of wheat allergy.

An exemplary list of celiac disease-associated T cell epitopes (recognized by CD4+ T cells) is provided in Table 1 herein below.

TABLE 1

| | | Peptide-binding register^{b} | | | | | | | | | |
|---|---|---|---|---|---|---|---|---|---|---|---|
| | Previous names | 1 | 2 | 3 | 4 | 5 | 6 | 7 | 8 | 9 | SEQ ID |
| Epitope^{a} | DQ2.5 restricted epitopes | | | | | | | | | | NO: |
| DQ2.5-glia-α1a | DQ2-α-I, α9 | P | F | P | Q | P | E | L | P | Y | 1 |
| DQ2.5-glia-α1b | DQ2-α-III | P | Y | P | Q | P | E | L | P | Y | 2 |
| DQ2.5-glia-α2 | DQ2-α-II, α2 | P | Q | P | E | L | P | Y | P | Q | 3 |
| DQ2.5-glia-α3 | glia-α20 | F | R | P | E | Q | P | Y | P | Q | 4 |
| DQ2.5-glia-γ1 | DQ2-γ-I | P | Q | Q | S | F | P | E | Q | Q | 5 |
| DQ2.5-glia-γ2 | DQ2-γ-II, γ30 | I | Q | P | E | Q | P | A | Q | L | 6 |
| DQ2.5-glia-γ3 | DQ2-γ-III | Q | Q | P | E | Q | P | Y | P | Q | 7 |
| DQ2.5-glia-γ4a | DQ2-γ-IV | S | Q | P | E | Q | E | F | P | Q | 8 |
| DQ2.5-glia-γ4b | DQ2-γ-VIIc | P | Q | P | E | Q | E | F | P | Q | 9 |
| DQ2.5-glia-γ4c | DQ2-γ-VIIa | Q | Q | P | E | Q | P | F | P | Q | 10 |
| DQ2.5-glia-γ4d | DQ2-γ-VIIb | P | Q | P | E | Q | P | F | C | Q | 11 |
| DQ2.5-glia-γ5 | DQ2-γ-VI | Q | Q | P | F | P | E | Q | P | Q | 12 |
| DQ2.5-glia-ω1 | DQ2-ω-I | P | F | P | Q | P | E | Q | P | F | 13 |
| DQ2.5-glia-ω2 | DQ2-ω-II | P | Q | P | E | Q | P | F | P | W | 14 |
| DQ2.5-glut-L1 | glutenin-17 | P | F | S | E | Q | E | Q | P | V | 15 |
| DQ2.5-glut-L2 | glutenin-156 | F | S | Q | Q | Q | E | S | P | F | 16 |
| DQ2.5-hor-1 | Hor-α9, Hα9 | P | F | P | Q | P | E | Q | P | F | 17 |
| DQ2.5-hor-2 | Hor-α2, Hα2 | P | Q | P | E | Q | P | F | P | Q | 18 |
| DQ2.5-hor-3 | hor-I-DQ2 | P | I | P | E | Q | P | Q | P | Y | 19 |
| DQ2.5-sec-1 | Sec-α9, Sα9 | P | F | P | Q | P | E | Q | P | F | 20 |
| DQ2.5-sec-2 | Sec-α2, α2 | P | Q | P | E | Q | P | F | P | Q | 21 |
| DQ2.5-ave-1a | Av-α9A | P | Y | P | E | Q | E | E | P | F | 22 |
| DQ2.5-ave-1b | Av-α9B, 1490 | P | Y | P | E | Q | E | Q | P | F | 23 |
| DQ2.2-glut-L1 | glutenin-17 | P | F | S | E | Q | E | Q | P | V | 24 |
| DQ8-glia-α1 | DQ8-α-I | E | G | S | F | Q | P | S | Q | E | 25 |
| DQ8-glia-γ1a | DQ8-γ-Ia | E | Q | P | Q | Q | P | F | P | Q | 26 |
| DQ8-glia-γ1b | DQ8-γ-Ib | E | Q | P | Q | Q | P | Y | P | E | 27 |
| DQ8-glut-H1 | HMW-glutenin | Q | G | Y | Y | P | T | S | P | Q | 28 |
| DQ8.5-glia-α1 | DQ8-α-I | E | G | S | F | Q | P | S | Q | E | 29 |
| DQ8.5-glia-γ1 | | P | Q | Q | S | F | P | E | Q | E | 30 |
| DQ8.5-glut-H1 | HMW-glutenin | Q | G | Y | Y | P | T | S | P | Q | 31 |

^{a}In the epitope names, these short terms are used to denote the type of proteins that the epitopes derive from:
Glia-α = α-gliadin;
Glia-β = β-gliadin;
Glia-γ = γ-gliadin;
Glia-ω = ω-gliadin;
Glut-L = low molecular weight glutenin;
Glut-H = high molecular weight glutenin;
Hor = hordein;
Sec = secalin;
Ave = avenin.
^{b}Glutamate residues (E) formed by TG2-mediated deamidation which are important for recognition by T cells are shown in bold. Additional glutamine residues also targeted by TG2 are underlined.

As used herein, the term “T cell receptor” or “TCR” refers to a complex of membrane proteins that participate in the activation of T cells in response to the presentation of antigen. The TCR is responsible for recognizing antigens bound to major histocompatibility complex molecules. TCR is composed of a heterodimer of an alpha (α) and beta (β) chain, although in some cells the TCR consists of gamma and delta chains. TCRs may exist in alpha/beta and gamma/delta forms, which are structurally similar but have distinct anatomical locations and functions. Each chain is composed of two extracellular domains, a variable and constant domain. In some embodiments, the TCR may be modified on any cell comprising a TCR, including, for example, a helper T cell, a cytotoxic T cell, a memory T cell, regulatory T cell, natural killer T cell, and gamma delta T cell. TCRs in the present invention may exist in a variety of forms including different fragments of TCR with or without mutations.

The term “T cell immunogen” refers to an agent (for example a protein) that is capable of eliciting a T cell mediated immune response. A T cell immunogen comprises at least one epitope. In one embodiment, the T cell immunogen is a wheat protein, such as a gluten protein.

Gluten proteins are characterized in particular by their specific composition of amino acids, these proteins containing a high content of proline residues (approximately 30% of the total amino acid residues), and glutamine residues (approximately 40% of the total amino acid residues). The constituent proteins of gluten are also characterized by the presence of repeated amino acid sequences. According to a particular embodiment, the gliadin is an alpha-gliadin, a beta-gliadin, a gamma-gliadin, an omega-2 gliadin and an omega-5 gliadin. In another embodiment, the glutenin is a low-molecular-weight glutenin or a high-molecular-weight glutenin.

In some embodiments, the computational prediction relies on a machine-learning algorithm trained to recognize residue pairing preferences on a dataset of TCR/antigen complexes. In other embodiments, the computational prediction relies on a machine-learning algorithm trained to recognize residue pairing preferences on a dataset of MHCII/antigen complexes. In a particular embodiment, the MHCII is HLA-DQ2 (e.g. HLA-DQ2.5, 2.2 or 2.3) or HLA-DQ8 or HLA-DQ8.5. In other embodiments the computational prediction is trained on sequences of pairs of experimentally verified epitopes/MHCII (e.g. HLA-DQ2 or HLA-DQ8) complexes. In other embodiments the computational prediction is trained on sequences of pairs of experimentally verified epitopes/TCR complexes (wherein the TCRs are derived from a celiac patient). In other embodiments the computational predictions are based on high throughput analysis of libraries and/or peptide derived from wheat polypeptides and their interaction with TCRs from a celiac subject or their interaction with Celiac related HMCII proteins (e.g. HLA-DQ2 or HLA-DQ8). Thus, the invention is also directed to a database on a computer readable medium comprising sequence of known antigen binding regions (ABRs) of TCRs or of MHCIIs and the sequences of known antigenic polypeptides to which they bind. In preferred embodiments, epitope prediction is not based on the 3D structure of the antigen or antigen-binding region, but only on the sequence of the TCRs or MHCIIs and the sequence of the peptide.

Methods of isolating T cells and/or T cell receptors include cell sorting or sequencing of samples using TCR-specific primers. In other embodiments the T cell receptor is isolated from T cells derived from a celiac patient. Methods of sequencing antibodies and T cell receptors isolated from a subject are known in the art.

In some embodiments, the predicted epitope is validated. In some embodiments, the predicted epitope is validated experimentally.

In a particular embodiment, the peptides used for validation are demidated (post-translational deamidation of glutamine residues to glutamates in peptide sequences by tissue transglutaminase (tTG2) that improves peptide-MHC complex stability).

Methods of experimentally testing T cell epitopes are known in the art, including, for example, using an MHCII binding assay. The binding of a predicted epitope to MHCII may be assessed based on its ability to inhibit the binding of a labeled (e.g. radiolabeled) probe peptide to purified MHCII molecules. MHCII molecules can be purified by affinity chromatography. The MHCII molecules may be obtained from a blood sample of a celiac disease patient. After an incubation period, the bound and unbound labeled species can be separated, and their relative amounts can be determined by either size-exclusion gel-filtration chromatography or monoclonal antibody capture of MHC. The percent of bound radioactivity can then be determined. Detailed protocol for a MHC II binding assay to be used is described in Sidney et al. (Sidney J, 2013).

In some embodiments, the validating uses libraries and/or peptides derived from the wheat protein to assess the importance of specific amino acids in specific positions for binding. In some embodiments, the library comprises a library of mutations to a subset or all of the amino acid residues of the wheat polypeptide. In some embodiments, the library is a yeast display library. In some embodiments, the library is a phage display library.

In some embodiments, the methods described herein are directed to identifying one or more celiac disease-related epitopes on a wheat polypeptide, and further, mutating one or more amino acid residues of the wheat polypeptide—for example the alpha gliadin polypeptide, the wild-type having the amino acid sequence as set forth in SEQ ID NO: 32.

In some embodiments, the method comprises mutating one or more amino acid residues of the wheat polypeptide in one or more of the identified epitopes. In some embodiments, the method comprises mutating 1, 2, 3, 4, 5, 6, 7, 8, 9, 10, 11, 12, 13, 14, 15, 16, 17, 18, 19, 20, 21, 22, 23, 24, or 25 or more amino acid residues of the polypeptide. In some embodiments, the one or more mutations destroy one or more (or all) of the identified epitopes on the polypeptide. Methods for making polypeptides comprising one or mutations are well known to one of ordinary skill in the art. In some embodiments, the one or more mutations are conservative mutations. In some embodiments, the one or more mutations are non-conservative mutations. In some embodiments, the one or more mutations are a mixture of conservative and non-conservative mutations.

The mutation of this aspect of the present invention may be a substitution, a deletion or an insertion.

The wheat polypeptide of this aspect of the present invention may be deamidated. Thus, for example, a wheat protein having an amino acid sequence as set forth in SEQ ID NO: 32 may be deamidated on position 66, 73 and/or 80. In other embodiments, the wheat polypeptide of this aspect of the present invention may be deamidated at any naturally occurring glutamine.

In one embodiment, the mutation of the alpha gliadin protein is such that the amino acid sequence of the mutated (and optionally glutamine deamidated) protein comprises the sequence as set forth in SEQ ID NOs: 36, 37, 38, 41, 42, 43, 46, 47 or 48.

According to a specific embodiment, the mutation does not affect the function of the wheat polypeptide.

Methods of introducing nucleic acid alterations to a gene of interest are well known in the art [see for example Menke D. Genesis (2013) 51:-618; Capecchi, Science (1989) 244:1288-1292; Santiago et al. Proc Natl Acad Sci USA (2008) 105:5809-5814; International Patent Application Nos. WO 2014085593, WO 2009071334 and WO 2011146121; U.S. Pat. Nos. 8,771,945, 8,586,526, 6,774,279 and UP Patent Application Publication Nos. 20030232410, 20050026157, US20060014264; the contents of which are incorporated by reference in their entireties] and include targeted homologous recombination, site specific recombinases, PB transposases and genome editing by engineered nucleases. Agents for introducing nucleic acid alterations to a gene of interest can be designed publically available sources or obtained commercially from Transposagen, Addgene and Sangamo Biosciences. In some embodiments, the generation of the alterations in the sequences of the genes may be achieved by screening sequences of existing plants in search of an existing variant of the desired sequence. Then, this existing sequence will be introduced into the genome of the target genome by crossbreeding. In other embodiments the desired variations will be introduced by introducing random mutagenesis, followed by screening for variants where the desired mutations occurred, followed by crossbreeding.

Following is a description of various exemplary methods used to introduce nucleic acid alterations to a gene of interest and agents for implementing same that can be used according to specific embodiments of the present invention.

Genome Editing using engineered endonucleases—this approach refers to a reverse genetics method using artificially engineered nucleases to cut and create specific double-stranded breaks at a desired location(s) in the genome, which are then repaired by cellular endogenous processes such as, homology directed repair (HDS) and non-homologous end-joining (NFfEJ). NFfEJ directly joins the DNA ends in a double-stranded break, while HDR utilizes a homologous sequence as a template for regenerating the missing DNA sequence at the break point. In order to introduce specific nucleotide modifications to the genomic DNA, a DNA repair template containing the desired sequence must be present during HDR. Genome editing cannot be performed using traditional restriction endonucleases since most restriction enzymes recognize a few base pairs on the DNA as their target and the probability is very high that the recognized base pair combination will be found in many locations across the genome resulting in multiple cuts not limited to a desired location. To overcome this challenge and create site-specific single- or double-stranded breaks, several distinct classes of nucleases have been discovered and bioengineered to date. These include the meganucleases, Zinc finger nucleases (ZFNs), transcription-activator like effector nucleases (TALENs) and CRISPR/Cas system.

Meganucleases—Meganucleases are commonly grouped into four families: the LAGLIDADG family, the GIY-YIG family, the His-Cys box family and the HNH family. These families are characterized by structural motifs, which affect catalytic activity and recognition sequence. For instance, members of the LAGLIDADG family are characterized by having either one or two copies of the conserved LAGLIDADG motif. The four families of meganucleases are widely separated from one another with respect to conserved structural elements and, consequently, DNA recognition sequence specificity and catalytic activity. Meganucleases are found commonly in microbial species and have the unique property of having very long recognition sequences (>14 bp) thus making them naturally very specific for cutting at a desired location. This can be exploited to make site-specific double-stranded breaks in genome editing. One of skill in the art can use these naturally occurring meganucleases, however the number of such naturally occurring meganucleases is limited. To overcome this challenge, mutagenesis and high throughput screening methods have been used to create meganuclease variants that recognize unique sequences. For example, various meganucleases have been fused to create hybrid enzymes that recognize a new sequence. Alternatively, DNA interacting amino acids of the meganuclease can be altered to design sequence specific meganucleases (see e.g., U.S. Pat. No. 8,021,867). Meganucleases can be designed using the methods described in e.g., Certo, M T et al. Nature Methods (2012) 9:073-975; U.S. Pat. No. 8,304,222; 8,021,867; 8,119,381; 8,124,369; 8,129,134; 8,133,697; 8,143,015; 8,143,016; 8,148,098; or 8,163,514, the contents of each are incorporated herein by reference in their entirety. Alternatively, meganucleases with site specific cutting characteristics can be obtained using commercially available technologies e.g., Precision Biosciences' Directed Nuclease Editor™ genome editing technology.

ZFNs and TALENs—Two distinct classes of engineered nucleases, zinc-finger nucleases (ZFNs) and transcription activator-like effector nucleases (TALENs), have both proven to be effective at producing targeted double-stranded breaks (Christian et al., 2010; Kim et al., 1996; Li et al., 2011; Mahfouz et al., 2011; Miller et al., 2010).

Basically, ZFNs and TALENs restriction endonuclease technology utilizes a non-specific DNA cutting enzyme which is linked to a specific DNA binding domain (either a series of zinc finger domains or TALE repeats, respectively). Typically a restriction enzyme whose DNA recognition site and cleaving site are separate from each other is selected. The cleaving portion is separated and then linked to a DNA binding domain, thereby yielding an endonuclease with very high specificity for a desired sequence. An exemplary restriction enzyme with such properties is Fokl. Additionally Fokl has the advantage of requiring dimerization to have nuclease activity and this means the specificity increases dramatically as each nuclease partner recognizes a unique DNA sequence. To enhance this effect, Fokl nucleases have been engineered that can only function as heterodimers and have increased catalytic activity. The heterodimer functioning nucleases avoid the possibility of unwanted homodimer activity and thus increase specificity of the double-stranded break.

Thus, for example to target a specific site, ZFNs and TALENs are constructed as nuclease pairs, with each member of the pair designed to bind adjacent sequences at the targeted site. Upon transient expression in cells, the nucleases bind to their target sites and the Fokl domains heterodimerize to create a double-stranded break. Repair of these double-stranded breaks through the nonhomologous end-joining (NHEJ) pathway most often results in small deletions or small sequence insertions. Since each repair made by NHEJ is unique, the use of a single nuclease pair can produce an allelic series with a range of different deletions at the target site. The deletions typically range anywhere from a few base pairs to a few hundred base pairs in length, but larger deletions have successfully been generated in cell culture by using two pairs of nucleases simultaneously (Carlson et al., 2012; Lee et al., 2010). In addition, when a fragment of DNA with homology to the targeted region is introduced in conjunction with the nuclease pair, the double-stranded break can be repaired via homology directed repair to generate specific modifications (Li et al., 2011; Miller et al., 2010; Urnov et al., 2005).

Although the nuclease portions of both ZFNs and TALENs have similar properties, the difference between these engineered nucleases is in their DNA recognition peptide. ZFNs rely on Cys2-His2 zinc fingers and TALENs on TALEs. Both of these DNA recognizing peptide domains have the characteristic that they are naturally found in combinations in their proteins. Cys2-His2 Zinc fingers typically found in repeats that are 3 bp apart and are found in diverse combinations in a variety of nucleic acid interacting proteins. TALEs on the other hand are found in repeats with a one-to-one recognition ratio between the amino acids and the recognized nucleotide pairs. Because both zinc fingers and TALEs happen in repeated patterns, different combinations can be tried to create a wide variety of sequence specificities. Approaches for making site-specific zinc finger endonucleases include, e.g., modular assembly (where Zinc fingers correlated with a triplet sequence are attached in a row to cover the required sequence), OPEN (low-stringency selection of peptide domains vs. triplet nucleotides followed by high-stringency selections of peptide combination vs. the final target in bacterial systems), and bacterial one-hybrid screening of zinc finger libraries, among others. ZFNs can also be designed and obtained commercially from e.g., Sangamo Biosciences™ (Richmond, Calif.).

Method for designing and obtaining TALENs are described in e.g. Reyon et al. Nature Biotechnology 2012 May; 30(5):460-5; Miller et al. Nat Biotechnol. (2011) 29: 143-148; Cermak et al. Nucleic Acids Research (2011) 39 (12): e82 and Zhang et al. Nature Biotechnology (2011) 29 (2): 149-53. A recently developed web-based program named Mojo Hand was introduced by Mayo Clinic for designing TAL and TALEN constructs for genome editing applications (can be accessed through www(dot)talendesign(dot)org). TALEN can also be designed and obtained commercially from e.g., Sangamo Biosciences™ (Richmond, Calif.).

CRISPR-Cas system—Many bacteria and archaea contain endogenous RNA-based adaptive immune systems that can degrade nucleic acids of invading phages and plasmids. These systems consist of clustered regularly interspaced short palindromic repeat (CRISPR) genes that produce RNA components and CRISPR associated (Cas) genes that encode protein components. The CRISPR RNAs (crRNAs) contain short stretches of homology to specific viruses and plasmids and act as guides to direct Cas nucleases to degrade the complementary nucleic acids of the corresponding pathogen. Studies of the type II CRISPR/Cas system of Streptococcus pyogenes have shown that three components form an RNA/protein complex and together are sufficient for sequence-specific nuclease activity: the Cas9 nuclease, a crRNA containing 20 base pairs of homology to the target sequence, and a trans-activating crRNA (tracrRNA) (Jinek et al. Science (2012) 337: 816-821.). It was further demonstrated that a synthetic chimeric guide RNA (gRNA) composed of a fusion between crRNA and tracrRNA could direct Cas9 to cleave DNA targets that are complementary to the crRNA in vitro. It was also demonstrated that transient expression of Cas9 in conjunction with synthetic gRNAs can be used to produce targeted double-stranded brakes in a variety of different species (Cho et al., 2013; Cong et al., 2013; DiCarlo et al., 2013; Hwang et al., 2013a,b; Jinek et al., 2013; Mali et al., 2013).

The CRIPSR/Cas system for genome editing contains two distinct components: a gRNA and an endonuclease e.g. Cas9.

The gRNA is typically a 20 nucleotide sequence encoding a combination of the target homologous sequence (crRNA) and the endogenous bacterial RNA that links the crRNA to the Cas9 nuclease (tracrRNA) in a single chimeric transcript. The gRNA/Cas9 complex is recruited to the target sequence by the base-pairing between the gRNA sequence and the complement genomic DNA. For successful binding of Cas9, the genomic target sequence must also contain the correct Protospacer Adjacent Motif (PAM) sequence immediately following the target sequence. The binding of the gRNA/Cas9 complex localizes the Cas9 to the genomic target sequence so that the Cas9 can cut both strands of the DNA causing a double-strand break. Just as with ZFNs and TALENs, the double-stranded brakes produced by CRISPR/Cas can undergo homologous recombination or NHEJ.

The Cas9 nuclease has two functional domains: RuvC and HNH, each cutting a different DNA strand. When both of these domains are active, the Cas9 causes double strand breaks in the genomic DNA.

A significant advantage of CRISPR/Cas is that the high efficiency of this system coupled with the ability to easily create synthetic gRNAs enables multiple genes to be targeted simultaneously. In addition, the majority of cells carrying the mutation present biallelic mutations in the targeted genes.

However, apparent flexibility in the base-pairing interactions between the gRNA sequence and the genomic DNA target sequence allows imperfect matches to the target sequence to be cut by Cas9.

Modified versions of the Cas9 enzyme containing a single inactive catalytic domain, either RuvC- or HNH-, are called ‘nickases’. With only one active nuclease domain, the Cas9 nickase cuts only one strand of the target DNA, creating a single-strand break or ‘nick’. A single-strand break, or nick, is normally quickly repaired through the HDR pathway, using the intact complementary DNA strand as the template. However, two proximal, opposite strand nicks introduced by a Cas9 nickase are treated as a double-strand break, in what is often referred to as a ‘double nick’ CRISPR system. A double-nick can be repaired by either NHEJ or HDR depending on the desired effect on the gene target. Thus, if specificity and reduced off-target effects are crucial, using the Cas9 nickase to create a double-nick by designing two gRNAs with target sequences in close proximity and on opposite strands of the genomic DNA would decrease off-target effect as either gRNA alone will result in nicks that will not change the genomic DNA.

Modified versions of the Cas9 enzyme containing two inactive catalytic domains (dead Cas9, or dCas9) have no nuclease activity while still able to bind to DNA based on gRNA specificity. The dCas9 can be utilized as a platform for DNA transcriptional regulators to activate or repress gene expression by fusing the inactive enzyme to known regulatory domains. For example, the binding of dCas9 alone to a target sequence in genomic DNA can interfere with gene transcription.

There are a number of publically available tools available to help choose and/or design target sequences as well as lists of bioinformatically determined unique gRNAs for different genes in different species such as the Feng Zhang lab's Target Finder, the Michael Boutros lab's Target Finder (E-CRISP), the RGEN Tools: Cas-OFFinder, the CasFinder: Flexible algorithm for identifying specific Cas9 targets in genomes and the CRISPR Optimal Target Finder.

In order to use the CRISPR system, both gRNA and Cas9 should be expressed in a target cell. The insertion vector can contain both cassettes on a single plasmid or the cassettes are expressed from two separate plasmids. CRISPR plasmids are commercially available such as the px330 plasmid from Addgene.

“Hit and run” or “in-out”—involves a two-step recombination procedure. In the first step, an insertion-type vector containing a dual positive/negative selectable marker cassette is used to introduce the desired sequence alteration. The insertion vector contains a single continuous region of homology to the targeted locus and is modified to carry the mutation of interest. This targeting construct is linearized with a restriction enzyme at a one site within the region of homology, electroporated into the cells, and positive selection is performed to isolate homologous recombinants. These homologous recombinants contain a local duplication that is separated by intervening vector sequence, including the selection cassette. In the second step, targeted clones are subjected to negative selection to identify cells that have lost the selection cassette via intrachromosomal recombination between the duplicated sequences. The local recombination event removes the duplication and, depending on the site of recombination, the allele either retains the introduced mutation or reverts to wild type. The end result is the introduction of the desired modification without the retention of any exogenous sequences.

The “double-replacement” or “tag and exchange” strategy—involves a two-step selection procedure similar to the hit and run approach, but requires the use of two different targeting constructs. In the first step, a standard targeting vector with 3′ and 5′ homology arms is used to insert a dual positive/negative selectable cassette near the location where the mutation is to be introduced. After electroporation and positive selection, homologously targeted clones are identified. Next, a second targeting vector that contains a region of homology with the desired mutation is electroporated into targeted clones, and negative selection is applied to remove the selection cassette and introduce the mutation. The final allele contains the desired mutation while eliminating unwanted exogenous sequences.

Site-Specific Recombinases—The Cre recombinase derived from the P1 bacteriophage and Flp recombinase derived from the yeast Saccharomyces cerevisiae are site-specific DNA recombinases each recognizing a unique 34 base pair DNA sequence (termed “Lox” and “FRY”, respectively) and sequences that are flanked with either Lox sites or FRT sites can be readily removed via site-specific recombination upon expression of Cre or Flp recombinase, respectively. For example, the Lox sequence is composed of an asymmetric eight base pair spacer region flanked by 13 base pair inverted repeats. Cre recombines the 34 base pair lox DNA sequence by binding to the 13 base pair inverted repeats and catalyzing strand cleavage and relegation within the spacer region. The staggered DNA cuts made by Cre in the spacer region are separated by 6 base pairs to give an overlap region that acts as a homology sensor to ensure that only recombination sites having the same overlap region recombine.

Basically, the site specific recombinase system offers means for the removal of selection cassettes after homologous recombination. This system also allows for the generation of conditional altered alleles that can be inactivated or activated in a temporal or tissue-specific manner. Of note, the Cre and Flp recombinases leave behind a Lox or FRT “scar” of 34 base pairs. The Lox or FRT sites that remain are typically left behind in an intron or 3′ UTR of the modified locus, and current evidence suggests that these sites usually do not interfere significantly with gene function.

Thus, Cre/Lox and Flp/FRT recombination involves introduction of a targeting vector with 3′ and 5′ homology arms containing the mutation of interest, two Lox or FRT sequences and typically a selectable cassette placed between the two Lox or FRT sequences. Positive selection is applied and homologous recombinants that contain targeted mutation are identified. Transient expression of Cre or Flp in conjunction with negative selection results in the excision of the selection cassette and selects for cells where the cassette has been lost. The final targeted allele contains the Lox or FRT scar of exogenous sequences.

Transposases—As used herein, the term “transposase” refers to an enzyme that binds to the ends of a transposon and catalyzes the movement of the transposon to another part of the genome.

As used herein the term “transposon” refers to a mobile genetic element comprising a nucleotide sequence which can move around to different positions within the genome of a single cell. In the process the transposon can cause mutations and/or change the amount of a DNA in the genome of the cell.

A number of transposon systems that are able to also transpose in cells e.g. vertebrates have been isolated or designed, such as Sleeping Beauty [Izsvák and Ivics Molecular Therapy (2004) 9, 147-156], piggyBac [Wilson et al. Molecular Therapy (2007) 15, 139-145], Tol2 [Kawakami et al. PNAS (2000) 97 (21): 11403-11408] or Frog Prince [Miskey et al. Nucleic Acids Res. December 1, (2003) 31(23): 6873-6881]. Generally, DNA transposons translocate from one DNA site to another in a simple, cut-and-paste manner. Each of these elements has their own advantages, for example, Sleeping Beauty is particularly useful in region-specific mutagenesis, whereas Tol2 has the highest tendency to integrate into expressed genes. Hyperactive systems are available for Sleeping Beauty and piggyBac. Most importantly, these transposons have distinct target site preferences, and can therefore introduce sequence alterations in overlapping, but distinct sets of genes. Therefore, to achieve the best possible coverage of genes, the use of more than one element is particularly preferred. The basic mechanism is shared between the different transposases, therefore we will describe piggyBac (PB) as an example.

PB is a 2.5 kb insect transposon originally isolated from the cabbage looper moth, Trichoplusia ni. The PB transposon consists of asymmetric terminal repeat sequences that flank a transposase, PBase. PBase recognizes the terminal repeats and induces transposition via a “cut-and-paste” based mechanism, and preferentially transposes into the host genome at the tetranucleotide sequence TTAA. Upon insertion, the TTAA target site is duplicated such that the PB transposon is flanked by this tetranucleotide sequence. When mobilized, PB typically excises itself precisely to reestablish a single TTAA site, thereby restoring the host sequence to its pretransposon state. After excision, PB can transpose into a new location or be permanently lost from the genome.

Typically, the transposase system offers an alternative means for the removal of selection cassettes after homologous recombination quit similar to the use Cre/Lox or Flp/FRT. Thus, for example, the PB transposase system involves introduction of a targeting vector with 3′ and 5′ homology arms containing the mutation of interest, two PB terminal repeat sequences at the site of an endogenous TTAA sequence and a selection cassette placed between PB terminal repeat sequences. Positive selection is applied and homologous recombinants that contain targeted mutation are identified. Transient expression of PBase removes in conjunction with negative selection results in the excision of the selection cassette and selects for cells where the cassette has been lost. The final targeted allele contains the introduced mutation with no exogenous sequences.

For PB to be useful for the introduction of sequence alterations, there must be a native TTAA site in relatively close proximity to the location where a particular mutation is to be inserted.

Genome editing using recombinant adeno-associated virus (rAAV) platform—this genome-editing platform is based on rAAV vectors which enable insertion, deletion or substitution of DNA sequences in the genomes of live mammalian cells. The rAAV genome is a single-stranded deoxyribonucleic acid (ssDNA) molecule, either positive- or negative-sensed, which is about 4.7 kb long. These single-stranded DNA viral vectors have high transduction rates and have a unique property of stimulating endogenous homologous recombination in the absence of double-strand DNA breaks in the genome. One of skill in the art can design a rAAV vector to target a desired genomic locus and perform both gross and/or subtle endogenous gene alterations in a cell. rAAV genome editing has the advantage in that it targets a single allele and does not result in any off-target genomic alterations. rAAV genome editing technology is commercially available, for example, the rAAV GENESIS™ system from Horizon™ (Cambridge, UK).

It will be appreciated that the agent can be a mutagen that causes random mutations.

The mutagens may be, but are not limited to, genetic, chemical or radiation agents. For example, the mutagen may be ionizing radiation, such as, but not limited to, ultraviolet light, gamma rays or alpha particles. Other mutagens may include, but not be limited to, base analogs, which can cause copying errors; deaminating agents, such as nitrous acid; intercalating agents, such as ethidium bromide; alkylating agents, such as bromouracil; transposons; natural and synthetic alkaloids; bromine and derivatives thereof; sodium azide; psoralen (for example, combined with ultraviolet radiation). The mutagen may be a chemical mutagen such as, but not limited to, ICR191, 1,2,7,8-diepoxy-octane (DEO), 5-azaC, N-methyl-N-nitrosoguanidine (MNNG) or ethyl methane sulfonate (EMS).

Methods for qualifying efficacy and detecting sequence alteration are well known in the art and include, but not limited to, DNA sequencing, electrophoresis, an enzyme-based mismatch detection assay and a hybridization assay such as PCR, RT-PCR, RNase protection, in-situ hybridization, primer extension, Southern blot, Northern Blot and dot blot analysis.

Sequence alterations in a specific gene can also be determined at the protein level using e.g. chromatography, electrophoretic methods, immunodetection assays such as ELISA and western blot analysis and immunohistochemistry.

In addition, one ordinarily skilled in the art can readily design a knock-in/knock-out construct including positive and/or negative selection markers for efficiently selecting transformed cells that underwent a homologous recombination event with the construct. Positive selection provides a means to enrich the population of clones that have taken up foreign DNA. Non-limiting examples of such positive markers include glutamine synthetase, dihydrofolate reductase (DHFR), markers that confer antibiotic resistance, such as neomycin, hygromycin, puromycin, and blasticidin S resistance cassettes. Negative selection markers are necessary to select against random integrations and/or elimination of a marker sequence (e.g. positive marker). Non-limiting examples of such negative markers include the herpes simplex-thymidine kinase (HSV-TK) which converts ganciclovir (GCV) into a cytotoxic nucleoside analog, hypoxanthine phosphoribosyltransferase (HPRT) and adenine phosphoribosytransferase (ARPT).

In some embodiments, the one or more mutations do not disrupt the function of the polypeptide (e.g., do not disrupt the function of the mutated polypeptide relative to the function of the corresponding un-mutated polypeptide). In some embodiments the one or more mutation do not disrupt the dough strengthening ability of the polypeptide. In some embodiments the one or more mutation do not disrupt the dough elasticity promoting ability of the polypeptide. In some embodiments the one or more mutation do not disrupt the dough rising promoting ability of the polypeptide. In some embodiments, the one or more mutations do not significantly affect the growth of the wheat (for example production of seeds, number of seeds, size of seeds). In some embodiments, the one or more mutations do not disrupt native protein-protein interactions of the polypeptide (e.g., the mutated polypeptide retains the ability to form substantially the same protein-protein interactions as the corresponding un-mutated polypeptide). In some embodiments, the one or more mutations do not disrupt the three-dimensional structure of the polypeptide (e.g., the mutated polypeptide retains substantially the same three-dimensional structure as the corresponding un-mutated polypeptide). In some embodiments, the one or more mutations do not disrupt the folding of the polypeptide (e.g., the mutated polypeptide retains substantially the same protein folding as the corresponding un-mutated polypeptide). In some embodiments, the one or more mutations do not disrupt the translation of the polypeptide (e.g., the mutated polypeptide is translated with the same timing, at the same rate, to the same levels, etc. as the corresponding un-mutated polypeptide). In some embodiments, the one or more mutations do not disrupt the normal cellular localization of the polypeptide (e.g., the mutated polypeptide retains substantially the same cellular localization as the corresponding un-mutated polypeptide). In some embodiments, the one or more mutations do not disrupt any post-translational modifications on the polypeptide (e.g., the mutated polypeptide retains substantially the same post-translational modification profile as the corresponding un-mutated polypeptide). In still some embodiments, the one or more mutations do not disrupt the allergenicity of the wheat polypeptide (e.g., the mutated polypeptide retains substantially the same IgE antibody binding affinity as the corresponding un-mutated polypeptide).

Methods for checking the protein structure/fold/biochemical-biophysical properties of the de-epitoped gluten of the present invention include hydrodynamic studies (see for example Field, J. M., Tatham, A. S. & Shewry, P. R. 1987. Biochem. J. 247, 215-221; Castellia, F. et al., 2000. Thermochimica Acta 346, 153-160); NMR spectroscopy (see for example Bekkers, A. C., et al. 1996, In Gluten 96—Proc. 6th Int. Wheat Gluten Workshop, Sydney, September 1996 pp. 190-194. North Melbourne, Australia: Royal Australian Chemical Institute; Eliezer, D., Biophysical characterization of intrinsically disordered proteins. Curr Opin Struct Biol. 2009; 19(1):23-30); Circular dichroism measurements (see for example Tatham, A. S., Shewry, P. R., 1985. J. Cereal Sci. 3, 104-113); Heterologous expression analysis (see for example Tatham, A. S., Shewry, P. R., 1985. J. Cereal Sci. 3, 104-113); Static and dynamic light scattering measurements (see for example Herrera, M.; Dodero, V. In Proceedings of the F.Bioact. Process. Qual. & Nutr., 10-12 Apr. 2013; Sciforum Electronic Conferences Series; T. A. Egorov, FEBS Letters, Volume 434, Issues 1-2, 1998, Pages 215-217); Small-angle X-ray scattering (see for example Neil H. Thomson Biochimica et Biophysica Acta (BBA)—Protein Structure and Molecular Enzymology, Volume 1430, Issue 2, 1999, Pages 359-366; Eliezer, D., Curr Opin Struct Biol. 2009; 19(1):23-30); very-small-angle Neutron scattering (see for example Mohsen Dahesh et al., The Journal of Physical Chemistry B 2014 118 (38), 11065-11076. DOI: 10.1021/jp5047134; Gibbs, B. E. & Showalter, S. A. 2015, Biochemistry 54, 1314-1326; fluorescence correlation spectroscopy (FCS) (see for example Eliezer, D., Curr Opin Struct Biol. 2009; 19(1):23-3); and Single-Molecule FRET (smFRET) (see for example Gibbs, B. E. & Showalter, S. A. 2015, Biochemistry 54, 1314-1326). The contents of all the above described references are incorporated herein by reference.

Preferably, the mutated (i.e. de-epitoped) polypeptide binds with a poorer affinity to celiac related HMCII proteins (e.g. HLA-DQ2 or HLA-DQ8) or to T-cells derived from a celiac patient than a corresponding non-mutated polypeptide binds to T cells derived from the same celiac patient. Thus, the affinity value, measured in units of concentration, is at least 10%, 20%, 30%, 40%, 50%, 60%, 70% 80%, 90% or 100% higher for the de-epitoped polypeptide to celiac related HMCII proteins (e.g. HLA-DQ2 or HLA-DQ8) or to T-cells derived from a celiac patient than a corresponding non-mutated polypeptide binds to T cells derived from the same celiac patient. In one embodiment, the binding of the mutated (i.e. de-epitoped) polypeptide to Celiac related HMCII proteins (e.g. HLA-DQ2 or HLA-DQ8) or to T cells, is abrogated. Methods of measuring the binding of peptides/polypeptides to Celiac related HMCII proteins (e.g. HLA-DQ2 or HLA-DQ8) or to T cells are known in the art and include for example: 1) detection of peptide/MHCII complexes using a combination of gel-filtration and competitive binding to a well-defined radio-labeled reference peptide (Sidney et al., Curr. Protoc. Immunol. 2013); 2) Using MHCII tetramers with gluten peptides fusion to detect and quantify binding to gluten-specific CD4+ T cells by flow cytometer (Raki et al., PNAS 2007); 3) ELISpot assay to measure activation of gluten-specific CD4+ T cells by probing secretion of IFN-γ (Anderson et al., Gut 2005); 4) Proliferation assays of gluten-specific T cells in the presence of relevant APCs (e.g., HLA DQ8 or HLA DQ2.5 expressing cells) and gluten peptides (Kooy-Winkelaar et al., J. Immunol. 2011).

Preferably, the mutated (i.e. de-epitoped) polypeptide activates T-cells derived from a celiac patient to a lesser extent (e.g. by at least 10%, 20%, 30%, 40%, 50%, 60%, 70% 80%, 90% or 100%) than a corresponding non-mutated activates T cells derived from said celiac patient. An exemplary T cell activation assay is described in the Examples section herein below.

Thus, according to another aspect of the present invention there is provided an isolated glutenin or gliadin polypeptide being mutated compared to the corresponding wild-type glutenin or gliadin polypeptide such that it binds with a lower affinity to Celiac related HMCII proteins (e.g. HLA-DQ2 or HLA-DQ8) or T-cells derived from a celiac patient than a corresponding non-mutated polypeptide binds to Celiac related HMCII proteins (e.g. HLA-DQ2 or HLA-DQ8) or T cells derived from said celiac patient.

According to a particular embodiment, the glutenin or gliadin polypeptide is a recombinant polypeptide.

The present inventors further contemplate isolated polynucleotides which encode the above described glutenin or gliadin polypeptides. Such polynucleotides may be used to express the above described de-epitoped glutenin or gliadin polypeptides in host cells (e.g. bacteria or plants).

As used herein, the terms “polynucleotide”, “nucleic acid sequence”, “nucleic acid”, and variations thereof shall be generic to polydeoxyribonucleotides (containing 2-deoxy-D-ribose), to polyribonucleotides (containing D-ribose), to any other type of polynucleotide that is an N-glycoside of a purine or pyrimidine base, and to other polymers containing non-nucleotidic backbones, provided that the polymers contain nucleobases in a configuration that allows for base pairing and base stacking, as found in DNA and RNA. Thus, these terms include known types of nucleic acid sequence modifications, for example, substitution of one or more of the naturally occurring nucleotides with an analog, and inter-nucleotide modifications.

Commonly used expression systems for heterologous protein production include bacterial cells (e.g. E. coli), fungal cells (e.g. S. cerevisiae cells), plant cells (e.g. tobacco), insect cells (lepidopteran cells) and other mammalian cells (Chinese Hamster Ovary cells).

Expressing the exogenous polynucleotide of the present invention within a host cell (e.g. plant) can be effected by transforming one or more cells of the host with the exogenous polynucleotide.

Preferably, the transformation is effected by introducing to the host cell a nucleic acid construct which includes the exogenous polynucleotide of the present invention and at least one promoter capable of directing transcription of the exogenous polynucleotide in the host cell. Further details of suitable transformation approaches are provided hereinbelow.

As used herein, the term “promoter” refers to a region of DNA which lies upstream of the transcriptional initiation site of a gene to which RNA polymerase binds to initiate transcription of RNA. The promoter controls where (e.g., which portion of a plant, which organ within an animal, etc.) and/or when (e.g., which stage or condition in the lifetime of an organism) the gene is expressed.

Any suitable promoter sequence can be used by the nucleic acid construct of the present invention. Preferably the promoter is a constitutive promoter, a tissue-specific promoter or a plant-specific promoter (such as a wheat promoter).

Suitable constitutive promoters include, for example, CaMV 35S promoter (SEQ ID NO: 19; Odell et al., Nature 313:810-812, 1985); maize Ubi 1 (Christensen et al., Plant Sol. Biol. 18:675-689, 1992); rice actin (McElroy et al., Plant Cell 2:163-171, 1990); rice glutelin (Qu, Le Qing et al. J Exp Bot 59:9, 2417-2424, 2008); pEMU (Last et al., Theor. Appl. Genet. 81:581-588, 1991); and Synthetic Super MAS (Ni et al., The Plant Journal 7: 661-76, 1995). Other constitutive promoters include those in U.S. Pat. Nos. 5,659,026, 5,608,149; 5,608,144; 5,604,121; 5,569,597: 5,466,785; 5,399,680; 5,268,463; and 5,608,142.

Suitable tissue-specific promoters include, but not limited to, leaf-specific promoters such as described, for example, by Yamamoto et al., Plant J. 12:255-265, 1997; Kwon et al., Plant Physiol. 105:357-67, 1994; Yamamoto et al., Plant Cell Physiol. 35:773-778, 1994; Gotor et al., Plant J. 3:509-18, 1993; Orozco et al., Plant Mol. Biol. 23:1129-1138, 1993; and Matsuoka et al., Proc. Natl. Acad. Sci. USA 90:9586-9590, 1993.

Suitable wheat specific promoters include, but not limited to those described in Smirnova, O. G. and Kochetov, A. V. Russ J Genet Appl Res (2012) 2: 434. www(dot)doi(dot)org/10.1134/S2079059712060123.

The nucleic acid construct of the present invention preferably further includes an appropriate selectable marker and/or an origin of replication. Preferably, the nucleic acid construct utilized is a shuttle vector, which can propagate both in E. coli (wherein the construct comprises an appropriate selectable marker and origin of replication) and be compatible for propagation in cells. The construct according to the present invention can be, for example, a plasmid, a bacmid, a phagemid, a cosmid, a phage, a virus or an artificial chromosome.

Various methods can be used to introduce the expression vector of some embodiments of the invention into stem cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

The nucleic acid construct of the present invention can be utilized to stably or transiently transform plant cells. In stable transformation, the exogenous polynucleotide of the present invention is integrated into the plant genome and as such it represents a stable and inherited trait. In transient transformation, the exogenous polynucleotide is expressed by the cell transformed but it is not integrated into the genome and as such it represents a transient trait.

There are various methods of introducing foreign genes into both monocotyledonous and dicotyledonous plants (Potrykus, I., Annu. Rev. Plant. Physiol., Plant. Mol. Biol. (1991) 42:205-225; Shimamoto et al., Nature (1989) 338:274-276).

The principle methods of causing stable integration of exogenous DNA into plant genomic DNA include two main approaches:

(i) Agrobacterium-mediated gene transfer: Klee et al. (1987) Annu. Rev. Plant Physiol. 38:467-486; Klee and Rogers in Cell Culture and Somatic Cell Genetics of Plants, Vol. 6, Molecular Biology of Plant Nuclear Genes, eds. Schell, J., and Vasil, L. K., Academic Publishers, San Diego, Calif. (1989) p. 2-25; Gatenby, in Plant Biotechnology, eds. Kung, S. and Arntzen, C. J., Butterworth Publishers, Boston, Mass. (1989) p. 93-112.

(ii) Direct DNA uptake: Paszkowski et al., in Cell Culture and Somatic Cell Genetics of Plants, Vol. 6, Molecular Biology of Plant Nuclear Genes eds. Schell, J., and Vasil, L. K., Academic Publishers, San Diego, Calif. (1989) p. 52-68; including methods for direct uptake of DNA into protoplasts, Toriyama, K. et al. (1988) Bio/Technology 6:1072-1074. DNA uptake induced by brief electric shock of plant cells: Zhang et al. Plant Cell Rep. (1988) 7:379-384. Fromm et al. Nature (1986) 319:791-793. DNA injection into plant cells or tissues by particle bombardment, Klein et al. Bio/Technology (1988) 6:559-563; McCabe et al. Bio/Technology (1988) 6:923-926; Sanford, Physiol. Plant. (1990) 79:206-209; by the use of micropipette systems: Neuhaus et al., Theor. Appl. Genet. (1987) 75:30-36; Neuhaus and Spangenberg, Physiol. Plant. (1990) 79:213-217; glass fibers or silicon carbide whisker transformation of cell cultures, embryos or callus tissue, U.S. Pat. No. 5,464,765 or by the direct incubation of DNA with germinating pollen, DeWet et al. in Experimental Manipulation of Ovule Tissue, eds. Chapman, G. P. and Mantell, S. H. and Daniels, W. Longman, London, (1985) p. 197-209; and Ohta, Proc. Natl. Acad. Sci. USA (1986) 83:715-719.

The Agrobacterium system includes the use of plasmid vectors that contain defined DNA segments that integrate into the plant genomic DNA. Methods of inoculation of the plant tissue vary depending upon the plant species and the Agrobacterium delivery system. A widely used approach is the leaf disc procedure which can be performed with any tissue explant that provides a good source for initiation of whole plant differentiation. Horsch et al. in Plant Molecular Biology Manual A5, Kluwer Academic Publishers, Dordrecht (1988) p. 1-9. A supplementary approach employs the Agrobacterium delivery system in combination with vacuum infiltration. The Agrobacterium system is especially viable in the creation of transgenic dicotyledonous plants.

There are various methods of direct DNA transfer into plant cells. In electroporation, the protoplasts are briefly exposed to a strong electric field. In microinjection, the DNA is mechanically injected directly into the cells using very small micropipettes. In microparticle bombardment, the DNA is adsorbed on microprojectiles such as magnesium sulfate crystals or tungsten particles, and the microprojectiles are physically accelerated into cells or plant tissues.

Following stable transformation plant propagation is exercised. The most common method of plant propagation is by seed. Regeneration by seed propagation, however, has the deficiency that due to heterozygosity there is a lack of uniformity in the crop, since seeds are produced by plants according to the genetic variances governed by Mendelian rules. Basically, each seed is genetically different and each will grow with its own specific traits. Therefore, it is preferred that the transformed plant be produced such that the regenerated plant has the identical traits and characteristics of the parent transgenic plant. Therefore, it is preferred that the transformed plant be regenerated by micropropagation which provides a rapid, consistent reproduction of the transformed plants.

Micropropagation is a process of growing new generation plants from a single piece of tissue that has been excised from a selected parent plant or cultivar. This process permits the mass reproduction of plants having the preferred tissue expressing the fusion protein. The new generation plants which are produced are genetically identical to, and have all of the characteristics of, the original plant. Micropropagation allows mass production of quality plant material in a short period of time and offers a rapid multiplication of selected cultivars in the preservation of the characteristics of the original transgenic or transformed plant. The advantages of cloning plants are the speed of plant multiplication and the quality and uniformity of plants produced.

Micropropagation is a multi-stage procedure that requires alteration of culture medium or growth conditions between stages. Thus, the micropropagation process involves four basic stages: Stage one, initial tissue culturing; stage two, tissue culture multiplication; stage three, differentiation and plant formation; and stage four, greenhouse culturing and hardening. During stage one, initial tissue culturing, the tissue culture is established and certified contaminant-free. During stage two, the initial tissue culture is multiplied until a sufficient number of tissue samples are produced to meet production goals. During stage three, the tissue samples grown in stage two are divided and grown into individual plantlets. At stage four, the transformed plantlets are transferred to a greenhouse for hardening where the plants' tolerance to light is gradually increased so that it can be grown in the natural environment.

Although stable transformation is presently preferred, transient transformation of leaf cells, meristematic cells or the whole plant is also envisaged by the present invention.

Transient transformation can be effected by any of the direct DNA transfer methods described above or by viral infection using modified plant viruses.

Viruses that have been shown to be useful for the transformation of plant hosts include CaMV, TMV and BV. Transformation of plants using plant viruses is described in U.S. Pat. No. 4,855,237 (BGV), EP-A 67,553 (TMV), Japanese Published Application No. 63-14693 (TMV), EPA 194,809 (BV), EPA 278,667 (BV); and Gluzman, Y. et al., Communications in Molecular Biology: Viral Vectors, Cold Spring Harbor Laboratory, New York, pp. 172-189 (1988). Pseudovirus particles for use in expressing foreign DNA in many hosts, including plants, is described in WO 87/06261.

Preferably, the virus of the present invention is avirulent and thus is incapable of causing severe symptoms such as reduced growth rate, mosaic, ring spots, leaf roll, yellowing, streaking, pox formation, tumor formation and pitting. A suitable avirulent virus may be a naturally occurring avirulent virus or an artificially attenuated virus. Virus attenuation may be effected by using methods well known in the art including, but not limited to, sub-lethal heating, chemical treatment or by directed mutagenesis techniques such as described, for example, by Kurihara and Watanabe (Molecular Plant Pathology 4:259-269, 2003), Gal-on et al. (1992), Atreya et al. (1992) and Huet et al. (1994).

Suitable virus strains can be obtained from available sources such as, for example, the American Type culture Collection (ATCC) or by isolation from infected plants. Isolation of viruses from infected plant tissues can be effected by techniques well known in the art such as described, for example by Foster and Tatlor, Eds. “Plant Virology Protocols: From Virus Isolation to Transgenic Resistance (Methods in Molecular Biology (Humana Pr), Vol 81)”, Humana Press, 1998. Briefly, tissues of an infected plant believed to contain a high concentration of a suitable virus, preferably young leaves and flower petals, are ground in a buffer solution (e.g., phosphate buffer solution) to produce a virus infected sap which can be used in subsequent inoculations.

Construction of plant RNA viruses for the introduction and expression of non-viral exogenous polynucleotide sequences in plants is demonstrated by the above references as well as by Dawson, W. O. et al., Virology (1989) 172:285-292; Takamatsu et al. EMBO J. (1987) 6:307-311; French et al. Science (1986) 231:1294-1297; and Takamatsu et al. FEBS Letters (1990) 269:73-76.

When the virus is a DNA virus, suitable modifications can be made to the virus itself. Alternatively, the virus can first be cloned into a bacterial plasmid for ease of constructing the desired viral vector with the foreign DNA. The virus can then be excised from the plasmid. If the virus is a DNA virus, a bacterial origin of replication can be attached to the viral DNA, which is then replicated by the bacteria. Transcription and translation of this DNA will produce the coat protein which will encapsidate the viral DNA. If the virus is an RNA virus, the virus is generally cloned as a cDNA and inserted into a plasmid. The plasmid is then used to make all of the constructions. The RNA virus is then produced by transcribing the viral sequence of the plasmid and translation of the viral genes to produce the coat protein(s) which encapsidate the viral RNA.

Construction of plant RNA viruses for the introduction and expression in plants of non-viral exogenous polynucleotide sequences such as those included in the construct of the present invention is demonstrated by the above references as well as in U.S. Pat. No. 5,316,931.

Techniques for inoculation of viruses to plants may be found in Foster and Taylor, eds. “Plant Virology Protocols: From Virus Isolation to Transgenic Resistance (Methods in Molecular Biology (Humana Pr), Vol 81)”, Humana Press, 1998; Maramorosh and Koprowski, eds. “Methods in Virology” 7 vols, Academic Press, New York 1967-1984; Hill, S. A. “Methods in Plant Virology”, Blackwell, Oxford, 1984; Walkey, D. G. A. “Applied Plant Virology”, Wiley, N.Y., 1985; and Kado and Agrawa, eds. “Principles and Techniques in Plant Virology”, Van Nostrand-Reinhold, N.Y.

Mature plants generated from the transformed cells may then be cultivated under conditions suitable for expressing the exogenous polynucleotide within the mature plant.

In one embodiment, the plant host cell in which the expression construct is transfected does not naturally express gluten polypeptides (i.e. derived from a gluten-free plant). Thus, in one embodiment, the host cell is selected from the group consisting of amaranth, buckwheat, rice (brown, white, wild), corn millet, quinoa, sorghum, Montina, Job's tears and teff.

In another embodiment, the plant host cell in which the expression construct is transfected expresses wild-type gluten polypeptides. Such host cells include but are not limited to wheat varieties such as spelt, kamut, farro and durum, bulgar, semolina, barley, rye, triticale, Triticum (wheat cultivars—fielder, spelling, bobwhite, cheyenne, chinse spring and mjoelner) and oats. It will be appreciated that in host cells that naturally express gluten polypeptides, the present inventors further contemplate down-regulating expression thereof. Methods of down-regulating expression of naturally occurring gluten polypeptides are known in the art and include for example the use of RNA silencing agent and DNA editing agents. Examples of RNA silencing agents include, but are not limited to siRNA, miRNA, antisense molecules, DNAzyme, RNAzyme. One method of downregulating expression of gluten polypeptides has been described in Sanchez-Leon, Susana et al. “Low-gluten, Nontransgenic Wheat Engineered with CRISPR/Cas9.” Plant Biotechnology Journal 16.4 (2018): 902-910. PMC, the contents of which are incorporated herein by reference.

For generation of recombinant polypeptides, the present invention contemplates expression constructs that include sequences engineered to enhance stability, production, purification, yield or toxicity of the expressed peptide. For example, the expression of a fusion protein or a cleavable fusion protein comprising the mutated gluten protein of some embodiments of the invention and a heterologous protein can be engineered. Such a fusion protein can be designed so that the fusion protein can be readily isolated by affinity chromatography; e.g., by immobilization on a column specific for the heterologous protein. Where a cleavage site is engineered between the mutated gluten protein and the heterologous protein, the mutated gluten protein can be released from the chromatographic column by treatment with an appropriate enzyme or agent that disrupts the cleavage site [e.g., see Booth et al. (1988) Immunol. Lett. 19:65-70; and Gardella et al., (1990) J. Biol. Chem. 265:15854-15859].

Recovery of the recombinant polypeptide is effected following an appropriate time in culture. The phrase “recovering the recombinant polypeptide” refers to collecting the whole fermentation medium containing the polypeptide and need not imply additional steps of separation or purification. Notwithstanding the above, polypeptides of some embodiments of the invention can be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromatofocusing and differential solubilization.

The present inventors contemplate using the de-epitoped gluten polypeptides described herein for the preparation of foods suitable for consumption by a subject having celiac disease.

In one embodiment, the de-epitoped gluten polypeptides can be used in the preparation of edible flour.

The term “flour” as used herein refers to a foodstuff which is a free-flowing powder, typically obtained by milling. Flour is most often used in bakery food products, such as breads, cakes, pastries etc., but also in other food products such as pasta, noodles, breakfast cereals and the like.

Examples of flours include bread flour, all-purpose flour, unbleached flour, self-raising flour, white flour, brown flour and semolina flour.

Thus, according to still another aspect of the present invention there is provided a flour derived from a gluten-free plant, comprising at least one de-epitoped glutenin or gliadin polypeptide.

Examples of plants (e.g. grains) from which the flour is derived include but are not limited to amaranth, buckwheat, rice (brown, white, wild). corn millet, quinoa, sorghum and teff.

In one embodiment, the gluten-free plant is transformed with the de-epitoped gluten polypeptides and a flour is generated therefrom (for example by grinding, mincing, milling etc.).

In another embodiment, a flour is generated from a gluten-free plant (for example by grinding, mincing, milling etc.) and at least one recombinant de-epitoped gluten polypeptide is added. The amount and variety of de-epitoped gluten polypeptides can be adjusted to change the quality of the flour or the dough generated therefrom. Thus, the present inventors contemplate use of the recombinant de-epitoped gluten polypeptides of the invention as dough improvers.

According to still another aspect a flour is generated from wheat which has been genetically modified to express at least one de-epitoped gluten polypeptide of the present invention. Preferably, the genetically modified wheat has been further manipulated such that expression of wild-type gluten polypeptides have been down-regulated or eliminated (as described herein above). It will be appreciated that the wheat of this aspect of the present invention may be used to generate other edible products such as beer.

The present inventors further contemplate generating dough from any of the flours described herein.

The term “dough” should be understood as having its commonly used meaning, namely, a composition comprising as minimal essential ingredients flour and a source of liquid, for example at least water that is subjected to kneading and shaping. The dough is characterized by its malleability.

The term “malleable” should be understood as defining the capacity of the dough for adaptive changes without necessary being easily broken and as such its pliability, elasticity and/or flexibility which thereby allows the subjecting of the dough to any one of the following processing steps: stretching, shaping, extending, sheeting, morphing, fitting, kneading, molding, modeling, or the like. The shaping of the dough may be by any instrument having predetermined shapes or by a rolling pin or by hand.

The dough may be characterized by at least one property selected from the group consisting of: a higher development time (DT), a lower stability time (S), a higher degree of softening (DS), a higher consistency (C) value and any combination thereof, as compared to a corresponding dough being absent of the de-epitoped glutenin or gliadin polypeptide.

The dough may further be characterized by at least one property selected from the group consisting of: a. higher rigidity relative to a corresponding dough being absent of the de-epitoped glutenin or gliadin polypeptide; b. higher stability to mechanical solicitations relative to a corresponding dough being absent of the de-epitoped glutenin or gliadin polypeptide; c. higher critical tension value relative to a corresponding dough being absent of the de-epitoped glutenin or gliadin polypeptide; d. a lower deformation capacity relative to a corresponding dough being absent of the de-epitoped glutenin or gliadin polypeptide; e. has higher plasticity relative to a corresponding dough being absent of the de-epitoped glutenin or gliadin polypeptide; and f. higher consistency relative to a corresponding dough being absent of the de-epitoped glutenin or gliadin polypeptide.

The dough of this aspect of the present invention can comprise additional components such as salt, a flavoring agent, vegetable or vegetable part, oil, vitamins and olives.

The dough may further comprise a leavening agent, examples of which include unpasteurized beer, buttermilk, ginger beer, kefir, sourdough starter, yeast, whey protein concentrate, yogurt, biological leaveners, chemical leaveners, baking soda, baking powder, baker's ammonia, potassium bicarbonate and any combination thereof.

Processed products generated from the doughs of this aspect of the present invention include, but are not limited to pan bread, a pizza bread crust, a pasta, a tortilla, a Panini bread, a pretzel, a pie and a sandwich bread product.

As used herein the term “about” refers to ±10%

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Comprehensive Mapping of Celiac Epitopes

Celiac epitopes will be mapped. The assessment is going to be based on the predicted ability of a peptide within the gene sequence to bind specific MHCII molecules. Epitope validation will be performed using MHC II binding assays.

Methodology for Example 1

Literature Search. An extensive and exhaustive literature search for all experimentally-validated celiac epitopes will be carried out.

Computational prediction. Mapping will be performed by using bioinformatic tools that predict immunogenic epitope sequences based on their ability to bind HLA class II genes HLA-DQ2 or HLA-DQ8. For each protein, all possible peptides (9-13 residues each) will be synthesized in their unmodified version or demidated version (post-translational deamidation of glutamine residues to glutamates in peptide sequences by tissue transglutaminase (tTG2) that improves peptide-MHC complex stability (Sollid L, 2012)). All peptide sequences will be analyzed for their potential to serve as T-cell epitopes, and candidates will be further screened by an MHC II binding assay. Prioritization for mapping will be given to gluten proteins with empirically-identified celiac epitopes, and ones that have been identified as essential to bread quality.

Epitope Validation. Computational prediction will be empirically validated using MHC II binding assay. The binding of each predicted epitope to MHC II will be assessed based on its ability to inhibit the binding of a radiolabeled probe peptide to purified MHC molecules. MHC II molecules will be purified by affinity chromatography, and peptides will be radiolabeled using the chloramine T method. After an incubation period, the bound and unbound radiolabeled species will be separated, and their relative amounts will be determined by either size-exclusion gel-filtration chromatography or monoclonal antibody capture of MHC. The percent of bound radioactivity will then be determined. Detailed protocol for the MHC II binding assay to be used is described in Sidney et al. (Sidney J, 2013).

### Example 2

### Abrogate Peptide Immunogenicity (“De-Epitoping”) while Maintaining Gene Product Expression and Folding

Overview. For the predicted epitopes identified, we will design a library that introduces nucleic acid variations in the positions predicted to bind the MHC II molecules HLA DQ2.5 or DQ8. We will then use this library to search for mutations that abrogate binding to HLA DQ2.5 or DQ8 using a method for library screening or selection like phage display library. We will use deep-sequencing to identify variants with abrogated binding to HLA DQ2.5 or DQ8 (using MHC II binding assay as described for Example 1) but with intact expression and folding using yeast surface display (YSD) library. In this context the YD library will be used to measure and assess expression and folding, not binding. Together with the binding screening described above, this will confirm that the de-epitoped protein is well expressed, well folded, stable, and does not bind MHC II. Importantly, most glutenins and some gliadins are unfolded and thus, are hard to express on the surface of yeast. For those proteins we will use Nickl coated plates and circular dichroism analysis for expression/folding analysis.

Methodology:

De-Epitoping:

Selecting positions for library design: For the predicted epitopes and for the known epitopes, we will select positions that are predicted to be crucial for MHC II binding. Briefly, we will predict HLA-peptide interaction for the WT and for specific mutants. The difference between the prediction will help identify promising mutations. Data from wild wheat strains with reduced toxicity will also be used to determine positions with potential impact on immunogenicity. The selection will be based on a combination score that will take into account: (i) score of the prediction, (ii) a multiple sequence alignment that will assess the conservation of the residues. A higher score will be given to less conserved residues. (iii) synergy with other putative substitutions within the same region of the protein. This way we will choose positions to be altered and variation to be introduced in each position. The final library will include in each altered position also the WT residue. Existing data shows that often a single mutation suffices to abrogate peptide-MHC binding.

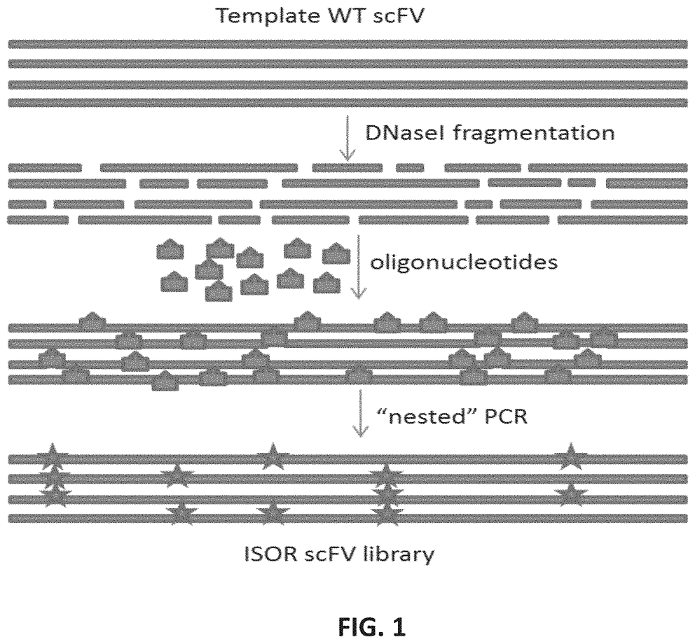

Library Design: We will order a library in which each position in the peptide (typically 9-13 amino acids in length, but may be shorter or longer, based on epitope mapping computational analysis) is replaced by other residues. Library design, based on selected positions analysis containing point mutations at selected positions will be generated by dubbed incorporation synthetic oligos via gene reassembly method (ISOR) (Herman 2007). Template gene (“WT”) based on the gene sequence will be ordered as a synthetic gene from IDT. Synthetic oligonucleotides containing the desired substitution and complementary to the appropriate DNA region will be ordered from IDT at low purification grade. All substitutions in the library will be encoded by a choice of codons that gives rise to the selected amino acids provided by the predictions, while minimizing the frequency of stop codons. The summary of the strategy is shown in FIG. 1. Briefly, template DNA will be amplified using reverse and forward primers in order to obtain microgram amounts of template. Next, DNA will be fragmented with DNaseI and fragments corresponded to 70-100 bp will be isolated. Next, DNA fragments will be mixed with various oligonucleotides amounts and PCR assembly reaction using Pfu Turbo DNA polymerase will be performed. The full length assembled genes will be further amplified by “nested” PCR using appropriate forward and reverse primers containing a DNA sequence recognized by specific restriction enzyme. DNA library of the desired diversity in pCTCON2 plasmid will be created by ligating digested pCTCON2 with digested pure “nested” PCR products and transforming electrocompetent E. coli cells with the purified ligation mix. Next, the complexity of the library will be assessed by sequencing random E. coli colonies. All plasmid containing cells will be pooled and EBY100 library will be isolated and saved.

Validation

Phage display library. Phage display involves the display of peptide libraries on the surfaces of bacteriophage F¢ episome, which allows M13 bacteriophage infection and propagation. Once introduced into the bacterial host, the DNA is resolved through DNA repair and replication, and the resulting library is packaged into phage particles. The DNA encapsulated by the positive phage clones (de-epitoped peptide sequences that do not bind HLA DQ2.5 or DQ8 as measured by MHC II binding test described above) is then used as template for deep sequencing. A detailed protocol can be found in Tonikian R, et al. 2007.

Expression and Folding Assessment

Yeast surface display (YSD): For gluten genes that fold and expressed well on the surface of yeast, YSD will be performed as previously described (Chao, G, 2006). Briefly, yeast library will be created at a diversity of about 1×10⁶ cells by transforming EBY100 cells with pCTCON2 plasmid library. Cells will be pooled and yeasts will be grown in SDCAA media containing pen/strep overnight. Next, cells will be collected by centrifuge and supplemented with SGCAA medium, which allows the expression of on the surface of yeast surface. The induction will be performed for 48 hours. Expressing cells will be isolated, analyzed and sorted by flow cytometry based on expression level. Plasmid will be isolated from positive clones and sequenced.

Deep sequencing. For YSD library, we will deep sequence the library and identify all de-epitoped gene variants that are expressed and folded correctly. We will then analyze the mutated gene sequences and assess which residue alteration in the epitope is predicted to lose binding to MHC II. Based on these results we will synthesize a de-epitoped gliadin or glutenin gene. For gluten genes that do not fold/expressed properly on the surface of yeast, candidate de-epitoped gene variants will be tested for expression using His₆-Tagged protein expression and nickel coated plates purification approach. Circular dichroism analysis will provide information on secondary structures in the protein.

Purification of His₆-tagged proteins using nickel coated plates. To investigate the expression of individual mutant proteins, a high throughput method for the purification of protein variants will be used. In this method protein purification procedure is based on the interaction between His₆-tagged proteins and Ni-NTA-coated microplates. A detailed protocol can be found in Lanio T, et al. 2000. Briefly, a plasmid vector pHis₆ that harbors the de-epitoped versions of a gluten gene will be generated. Transcription will be under the control of a combination of two lac-operators and a T7-promoter, which allows for effective repression or induction with IPTG. E. coli cells will be grown at 37° C. and transferred to LB medium. Expression of the variants will be induced by adding IPTG. After incubation, cells will be harvested by centrifugation and pellets will be resuspended in lysis buffer. The lysate will be transferred to Ni-NTA HisSorb and incubated with vortexing at room temperature. Plates will be washed with lysis buffer. His₆-tagged proteins will be eluted. Cell pellets from pre-cultures will be used to extract DNA from variant genes of interest using a standard DNA plasmid preparation or by PCR.

Circular dichroism analysis. Purified de-epitoped proteins that are adequately expressed will be further tested for folding using circular dichroism analysis as previously described (Srinivasan B, 2015). Purified protein will be dialyzed in acetic acid, and its circular dichroism spectra will be analyzed using a Spectropolarimeter. The far-UV circular dichroism spectra from 190 to 260 nm will be recorded in a 2-mm path length quartz cuvette with a resolution of 1 nm, a scan speed of 50 nm/min, and a protein concentration of 0.10 mg/mL. An average of three scans will be obtained. Mean residue ellipticity (degrees per square centimeter per decimole×10³) at a given wavelength will be calculated. Subsequent calculation of the contents of secondary structure will be performed using a specialized software. De-epitoped gene variants that preserve expression and folding (similar to unmodified counterparts) will be further validated for lack of immunogenicity using a T-cell activation assay.

Further Validation

T cell activation assay. Validation will be performed using an HLA-DQ-peptide tetramer-based assay. In this assay de-epitoped peptides or unmodified controls presented on HLA (DQ2.5 and DQ8) tetramers will be incubated with T-cells isolated from peripheral blood of CD patients (possibly under oral gluten challenge), or from fresh small intestinal biopsies that enable the culture of living cells obtained from the site of inflammation. T-cell binding and/or activation will be measured as previously described (Brottveit M, 2011). Complexes that show significant reduction or a complete abrogation of the binding and activation of these T cells will be selected for further assessment.

The specific alterations identified for each gliadin and glutenin will be introduced into the full gene sequence of the gene and will be used for functional testing as part of Example 3.

### Example 3

### Generate “Celiac-Safe” Gluten Protein Variants with Intact Biophysical Properties

Full gene sequences of de-epitoped gluten genes will be tested for preservation of their biophysical qualities. This will be done by recombinant expression of de-epitoped genes by any means, including but not restricted to, bacterial, viral or mammalian expression technologies. Purified recombinant de-epitoped gluten genes (single genes or in combination) will be added, in different quantities or combinations to gluten-free dough or flour or any other gluten-free product. Alternatively, flour/dough from crops other than wheat (e.g. rice flour) may be used, to attempt improvement of bread quality. The contribution of de-epitoped variant to bread/flour qualities such as mixing properties, rising, elasticity and strength of dough. Biophysical properties of de-epitoped variants will be compared to unmodified (“WY”) counterparts to validate comparable functionality.

Methodology:

Recombinant protein production. We will engineer an expression construct with molecular attributes (e.g., a strong promoter, an efficient ribosome binding site) optimized for a selected host. For bacterial expression, transformation of modified and unmodified gluten genes will be followed by screening studies and optimization of growth conditions (host, induction, media, temperature, additives) to drive either soluble or inclusion bodies expression. Expression assessment will be performed by SDS-PAGE/Coomassie or Western blot. Recombinant protein will then be purified from lysate fraction or inclusion bodies. For Baculovirus protein expression, genes will be subcloned into baculovirus expression vector and expressed in insect cells (e.g., SF9 or SF21). This will be followed by virus generation, amplification and cloning (limited dilution or plaque purification). High-titer viral stocks will be generated. Expression assessment will be done by Western blot or ELISA. Recombinant proteins will be purified from cell pellets. For mammalian protein expression, we will use mammalian cells (e.g. in CHO, HEK293, HEK293E) for recombinant expression of our vectors. Expression will be tested by western blot or ELISA isolated from cell lysates.

Validation. Similarly to the methods described in Example 1, we will conduct MHCII binding assays with recombinant proteins to validate the lack of immunogenicity (de-epitoped variants) or immunogenicity (WT variants).

Assessment of Biophysical Qualities of De-Epitoped Gluten Gene Sets.

We expect to test each recombinant protein (WT or de-epitoped variants) separately or in combination, to determine the contribution of individual proteins and specific combinations to different biophysical characteristics. We will test the addition of different quantities or concentrations and ratios between different recombinant proteins to achieve optimal dough and bread properties. Flour will be produced followed by dough preparation assessment of its biophysical properties. Testing include: rheological properties, stability, dough development time, peak consistency values, crumb color and texture attributes, resilience and adhesiveness. Protocols for testing will be adopted from Pătraşcu L, et al. 2017.

### Example 4

### Engineer a Plant to Express De-Epitoped Gluten Gene Variants with Intact Biophysical Properties

We will use the following approaches to express de-epitoped gene(s) in the plant:
1. Genome editing to modify the DNA sequence of target gluten genes in bread wheat (Triticum aestivum) using CRISPR/Cas9 approach.
2. Plant genetic engineering to express the de-epitoped gene under the control of the native promoter of the gene while silencing the expression of the native gene with artificial microRNAs (amiRNA).
2.1. Transformation of the de-epitoped gene under the control of its native promoter while silencing the expression of the native gene using RNAi approach or using deletion lines (in which the WT gene is not expressed) of Triticum aestivum.
   3. Transformation of de-epitoped gluten genes into other crops (e.g., rice, maize, etc.).

For all approaches, the unmodified (WT) version of the gene will serve as a baseline control. The objective is to ascertain the modifications made to the genes remain non-immunogenic when expressed in the plant, and do not negatively impact dough preparation and baking (as described in Example 3). In addition, for all approaches, we will assess growth of plants.

Methodology

WT gene Silencing in Wheat. We will express the de-epitoped gene under the control of the native promoter of the gene while silencing the expression of the native gene. To this end artificial microRNAs (amiRNA) will be designed to selectively target the native transcript that is ‘blind’ to de-epitoped gene using the WMD3-web microRNA designer (wmd3(dot)weigelworld(dot)org/cgi-bin/webapp(dot)cgi). The silencing efficiency will be tested prior to the transformation of the plant by screening between 2-5 amiRNA for their silencing efficiency using transient expression assay approach; the native and altered genes fused to two different reporter genes (GFP or luciferase in the two reciprocal possibilities) and controlled by strong constitutive promoter will be transiently co-expressed along with each designed amiRNA in leaves of Nicotiana benthamiana. The most efficient amiRNA will be continued to the next step of generating transgenic plants. The expression of the amiRNA will be controlled by a strong wheat-specific promoter. Both the de-epitoped gene (modified genomic fragment including the promoter, UTRs and introns) and the selected amiRNA will be cloned into the same binary vector. Transgenic plants will be generated by agrobacterium mediate transformation according to the efficient protocol (Ishida Y, 2015). The resulting transgenic wheat will be evaluated for silencing efficiency and expression levels of the altered gene using single nucleotide polymorphisms (SNPs) discriminating approach on cDNA; either derived cleaved amplified polymorphic sequences (dCAPS) or simple allele discriminating PCR (SAP) (Chum, P Y, 2012; Bui, M, 2009). Transgenic lines with maximal silencing of the native transcript that show good plant growth and non-disrupted development phenotype similar to the WT will be continued.

Cloning and Transformation: Gluten genes will be cloned from select wheat cultivar.

Glutenin genes Dx5 and Dy1 were previously reported to contribute to dough viscoelasticity (Rooke L, 1999; Popineau Y, 2001; Gadaleta, A, 2008). It has been previously reported that the highly immunogenic α2-gliadin locus on the short arm of chromosome 6D resulted in significant loss of dough functionality (Van den Broeck HC, 2009). Based on these data, we will transform the plant with Dx5 and Dy1 glutenins in combination with α2-gliadin to generate a gluten complex and serve as a baseline comparator in functionality assays.

Transgene expression in wheat: Immature embryos of healthy plants of wheat cultivar grown in a well-conditioned greenhouse will be pretreated with centrifuging and cocultivated with Agrobacterium tumefaciens under the protocol described by Ishida et al. (Ishida Y, 2015).

Transgene expression in rice: In general, cloning and transformation strategies will follow protocols described in Jo, et al. 2017. Genes will be inserted individually into an expression vector and expressed in the high-amylose Korean rice cultivar Koami (Oryza sativa L.) under the control of the rice endosperm-specific Glu-B1 promoter. The constructed vectors will be introduced into Agrobacterium tumefaciens (LBA4404) and genes of interest will be inserted into the genome of japonica-type Korean rice cultivar Koami.

Transgene expression in maize: Genes will be inserted individually into an expression vector and expressed in Maize (Zea mays L.), under the control of a maize endogenous promoter. Agrobacterium-mediated maize immature transformation will be performed based on a method developed by Ishida et al. (Ishida Y, 1996) to yield high frequency of transgenic event production.

For all transgenes cultivation will be performed and harvested transgenic seeds will be stored at a 4° C. Transgene expression will be characterized by SDS-PAGE, imaging or other molecular techniques for expression and localization analyses.

Validation. MHCII binding assays with extracts from transgenic seeds/plants will be conducted to validate the lack of immunogenicity of the variants expressed in the plant.

Assessment of Biophysical Qualities of De-Epitoped Gluten Gene Sets.

This will be performed similarly to the methods described for Example 3.

Genome editing: De-epitoped gluten genes that will exhibit the best performance in the transgenic wheat and immunological assays will be chosen for genome editing using the CRISPER/Cas9 approaches. We will use CRISPR/cas9 to remove the WT gluten gene from the wheat genome and replace it with the sequence of the de-epitoped gene. This will yield several cells, each of which contains a different version of the de-epitoped gene. A recent approach uses of DNA-free editing of bread wheat by delivering in vitro transcripts or ribonucleoprotein complexes of CRISPR/Cas9 by particle bombardment and may be used for this purpose (Liang Z, 2018). Genotyping genome-edited mutations in wheat using CRISPR ribonucleoprotein complexes will be done using the method described by Liang et al. (Liang Z, 2018a).

### Example 5

### Exemplary Gluten Peptides Showing Reduced Binding to MHC

Materials and Methods

Measurement of MHC/peptide interactions: Computational prediction algorithm was used to generate a list of putative non-binding peptides. Those peptides were synthesized and binding to MHC was measured as described in Sidney J et al, 2013. Briefly, competition assay using different concentrations of WT and modified gluten peptides were conducted by diluting the peptides in NP40 buffer, and incubation for 2-4 days with purified MHC and radiolabeled known MHC binding peptide. IC50 of WT and modified peptides was calculated. Validated gluten peptide epitopes were analyzed for MHC binding as a positive control. Some of the peptides were tested also in a deamidated form.

Results

Table 2 shows the IC50 measured for several variants that were predicted to have compromised binding to MHC. When values are greater than that of the native peptide, the binding of the engineered peptide chain is compromised with respect to the native gluten. For each peptide, the number of modifications with respect to the WT native peptide is listed.

TABLE 2

| | # of modi- | IC50 | |
|---|---|---|---|
| Variant | fication | (nM) | Sequence |
| P1a | native | 271 | LQLQPFPQPQLPYPQ - |
| | | | SEQ ID NO: 34 |
| P1b | deamidated | 14 | LQLQPFPQPELPYPQ - |
| | | | SEQ ID NO: 35 |
| P1d | 3 | 1614 | LQLQPFPHPELSYSQ - |
| | | | SEQ ID NO: 36 |
| P1f | 3 | 5439 | LQLQPFDQPRLPWPQ - |
| | | | SEQ ID NO: 37 |
| P1g | 3 | 11158 | LQLQPFWQPKLPGPQ - |
| | | | SEQ ID NO: 38 |
| P2a | native | 437 | QLPYPQPQLPYPQPQ - |
| | | | SEQ ID NO: 39 |
| P2b | deamidated | 56 | ELPYPQPELPYPQPQ - |
| | | | SEQ ID NO: 40 |
| P2c | 3 | 2208 | HLPYPQPELRYSQPQ - |
| | | | SEQ ID NO: 41 |
| P2f | 3 | 2584 | MLRYPQPWLPYPQPQ - |
| | | | SEQ ID NO: 42 |
| P2g | 4 | 3747 | KLPYPQGRLPGPQPQ - |
| | | | SEQ ID NO: 43 |
| P3a | native | 481 | PYPQPQLPYPQPQPF - |
| | | | SEQ ID NO: 44 |
| P3b | deamidated | 87 | PYPQPELPYPQPQPF - |
| | | | SEQ ID NO: 45 |
| P3c | 3 | 2993 | PYPHPRLPYTQPQPF - |
| | | | SEQ ID NO: 46 |
| P3d | 3 | 1991 | PYSQPESPYMQPQPF - |
| | | | SEQ ID NO: 47 |
| P3f | 4 | 879 | PYPHPWLRGPQPQPF - |
| | | | SEQ ID NO: 48 |

### Example 6

### Bread-Baking with Isolated Gluten Protein

The aim of this experiment was to demonstrate the feasibility of using purified gluten combined with non-wheat starch as a bread baking formula. The present inventors wanted to know whether addition of purified gluten to non-wheat starches can achieve the functionality and taste results of regular wheat. In order to do this, purified gluten in the quantities described below was added into a mix of cornstarch and rice flour as replacements of wheat starch. The mixture was mixed until dough with the right texture was achieved. The dough was then allowed to rise and then folded into both a bread pan for baking and a free form (not in a pan) for baking. The bread was then baked.

### Recipe Used:

| Ingredient | Amount | |
|---|---|---|
| Salt | 20 | gr |
| Margarine | 50 | gr |
| Fresh Yeasts | 45 | gr |
| Gluten | 350 | gr |
| S-500 (dough conditioners) | 10 | gr |
| Corn starch | 500 | gr |
| Rice flour | 500 | gr |
| Water | | |

Results

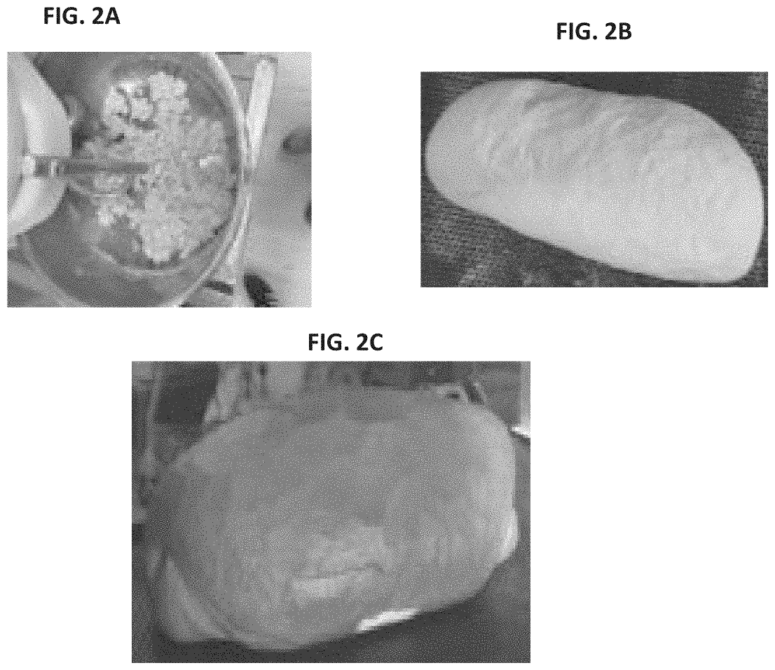

The resulting bread had the same texture and taste as wheat-based bread—see FIGS. 2A-C.

### REFERENCES

Brottveit M, Ráki M, Bergseng E, Fallang L E, Simonsen B, Løvik A, Larsen S, Løberg E M, Jahnsen F L, Sollid L M, Lundin K E. Assessing possible celiac disease by an HLA-DQ2-gliadin Tetramer Test. Am J Gastroenterol. 2011; 106(7):1318-24.
Bui M, Liu Z. Simple allele-discriminating PCR for cost-effective and rapid genotyping and mapping. Plant Methods. 2009; 5:1.
Chao G, Lau W L, Hackel B J, Sazinsky S L, Lippow S M, Wittrup K D. Isolating and engineering human antibodies using yeast surface display. Nat Protoc. 2006; 1(2):755-68.
Chum P Y, Haimes J D, André C P, Kuusisto P K, Kelley M L. Genotyping of plant and animal samples without prior DNA purification. J Vis Exp. 2012; (67).
Gadaleta, A.; Blechl, A. E.; Nguyen, S.; Cardone, M. F.; Ventura, M.; Quick, J. S.; Blanco, A. Stably expressed D-genome-derived HMW glutenin subunit genes transformed into different durum wheat genotypes change dough mixing properties. Mol. Breed. 2008, 22, 267-279.
Herman A, Tawfik D S. Incorporating Synthetic Oligonucleotides via Gene Reassembly (ISOR): a versatile tool for generating targeted libraries. Protein Eng Des Sel. 2007; 20(5):219-26.
Ishida Y, Saito H, Ohta S, Hiei Y, Komari T, Kumashiro T. High efficiency transformation of maize (Zea mays L.) mediated by Agrobacterium tumefaciens. Nat Biotechnol. 1996; 14(6):745 50.
Ishida Y, Tsunashima M, Hiei Y, Komari T. Wheat (Triticum aestivum L.) transformation using immature embryos. Methods Mol Biol. 2015; 1223:189-98.
Jo Y M, Cho K, Lee H J, Lim S H, Kim J S, Kim Y M, Lee J Y. Cellular Localization of Wheat High Molecular Weight Glutenin Subunits in Transgenic Rice Grain. Int J Mol Sci. 2017 Nov. 18; 18(11).
Lanio T, Jeltsch A, Pingoud A. Automated purification of His6-tagged proteins allows exhaustive screening of libraries generated by random mutagenesis. Biotechniques. 2000; 29(2):338-42.
Liang Z, Chen K, Zhang Y, Liu J, Yin K, Qiu J L, Gao C. Genome editing of bread wheat using biolistic delivery of CRISPR/Cas9 in vitro transcripts or ribonucleoproteins. Nat Protoc. 2018; 13(3):413-430.
Liang Z, Chen K, Yan Y, Zhang Y, Gao C. Genotyping genome-edited mutations in plants using CRISPR ribonucleoprotein complexes. Plant Biotechnol J. 2018. doi: 10.1111/pbi.12938. [Epub ahead of print]
Molberg O, Kett K, Scott H, Thorsby E, Sollid L M, Lundin K E. Gliadin specific, HLA DQ2-restricted T cells are commonly found in small intestinal biopsies from coeliac disease patients, but not from controls. Scand J Immunol. 1997; 46(3):103-109.
Pătraşu L, Banu I, Vasilean I, Aprodu I. Effect of gluten, egg and soy proteins on the rheological and thermo-mechanical properties of wholegrain rice flour. Food Sci Technol Int. 2017; 23(2):142-155.
Popineau, Y.; Deshayes, G.; Lefebvre, J.; Fido, R.; Tatham, A. S.; Shewry, P. R. Prolamin aggregation, gluten viscoelasticity, and mixing properties of transgenic wheat lines expressing 1Ax and 1Dx high molecular weight glutenin subunit transgenes. J. Agric. Food Chem. 2001, 49, 395-401.
Rooke, L.; Bekes, F.; Fido, R.; Barro, F.; Gras, P.; Tatham, A. S.; Barcelo, P.; Lazzeri, P.; Shewry, P. R. Overexpression of a gluten protein in transgenic wheat results in greatly increased dough strength. J. Cereal Sci. 1999, 30, 115-120.
Sidney J, Southwood S, Moore C, Oseroff C, Pinilla C, Grey H M, Sette A. Measurement of MHC/peptide interactions by gel filtration or monoclonal antibody capture. Curr Protoc Immunol. 2013; Chapter 18:Unit 18.3.
Sollid L M, Qiao S W, Anderson R P, Gianfrani C, Koning F. Nomenclature and listing of celiac disease relevant gluten T-cell epitopes restricted by HLA-D Q molecules. Immunogenetics. 2012; 64(6):455-60.
Srinivasan B, Focke-Tejkl M, Weber M, Pahr S, Baar A, Atreya R, Neurath M F, Vogelsang H, Huber W D, Valenta R. Usefulness of recombinant γ-gliadin 1 for identifying patients with celiac disease and monitoring adherence to a gluten-free diet. J Allergy Clin Immunol. 2015; 136(6):1607-1618.e3.
Tonikian R, Zhang Y, Boone C, Sidhu S S. Identifying specificity profiles for peptide recognition modules from phage-displayed peptide libraries. Nat Protoc. 2007; 2(6):1368-86.
Van den Broeck H C, van Herpen T W, Schuit C, Salentijn E M, Dekking L, Bosch D, Hamer R J, Smulders M J, Gilissen L J, van der Meer I M. Removing celiac disease-related gluten proteins from bread wheat while retaining technological properties: a study with Chinese Spring deletion lines. BMC Plant Biol. 2009 Apr. 7; 9:41.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

In addition, the priority document of this application is hereby incorporated herein by reference in its entirety.

1. A method for identifying an epitope of a wheat T cell immunogen, the method comprising identifying an epitope on the wheat T cell immunogen for the ability to bind a major histocompatibility complex (MHC) class II, thereby identifying the epitope of a wheat T cell immunogen.

2. A method for de-epitoping a wheat polypeptide, the method comprising mutating one or more amino acid residues of a celiac-associated epitope on the wheat polypeptide to generate a de-epitoped polypeptide having one or more mutation in said celiac-associated epitope, thereby de-epitoping the wheat polypeptide.

3. The method of claim 1, wherein said epitope is a celiac-associated epitope.

4. The method of claim 2, wherein said mutating one or more amino acids does not reduce the allergenicity of said wheat polypeptide.

5. The method of claim 2, wherein said wheat polypeptide is a glutenin or a gliadin.

6. (canceled)

7. The method of claim 2, further comprising identifying an epitope on the wheat polypeptide for the ability to bind a major histocompatibility complex (MHC) class II wherein said predicting is performed prior to said mutating.

8. (canceled)

9. The method of claim 2, further comprising identifying an epitope on the wheat polypeptide from the amino acid sequence of antigen binding regions of T cell receptors (TCRs) which bind to said wheat polypeptide.

10. (canceled)

11. The method of claim 1, wherein said identifying is by computationally predicting the epitope and is optionally performed using a machine-learning algorithm trained to recognize residue pairing preferences on a dataset of MHC II-peptide complexes.

12. The method of claim 1, wherein said identifying is by computationally predicting the epitope and is optionally performed using a machine-learning algorithm trained to recognize whether a given MHC II and a given T-cell immunogen are likely to bind each other based on a data set of MHC II-peptide interactions.

13. The method of claim 9, wherein said identifying is by computationally predicting the epitope and is performed using an algorithm trained to predict TCR-peptide interactions for peptides derived from said wheat polypeptide.

14-17. (canceled)

18. The method of claim 2, wherein said de-epitoped polypeptide binds with a lower affinity to T-cells derived from a celiac patient than a corresponding non-mutated polypeptide binds to T cells derived from said celiac patient and/or said de-epitoped polypeptide activates T-cells derived from a celiac patient to a lesser extent than a corresponding non-mutated polypeptide activates T cells derived from said celiac patient.

19-22. (canceled)

23. An isolated glutenin or gliadin polypeptide being mutated compared to the corresponding wild-type glutenin or gliadin polypeptide such that it binds with a lower affinity to T-cells derived from a celiac patient than a corresponding non-mutated polypeptide binds to T cells derived from said celiac patient.

24. An isolated polynucleotide encoding the isolated glutenin or gliadin polypeptide of claim 23.

25. An expression vector comprising the isolated polynucleotide of claim 24, operatively linked to a transcriptional regulatory sequence so as to allow expression of said glutenin or gliadin in a plant cell.

26. The expression vector of claim 25, wherein said transcriptional regulatory sequence comprises a plant promoter.

27. The expression vector of claim 26, wherein said plant promoter comprises a wheat promoter.

28. A flour derived from a gluten-free plant, comprising a de-epitoped glutenin or gliadin polypeptide.

29. The flour of claim 28, wherein said de-epitoped glutenin or gliadin polypeptide is mutated compared to the corresponding wild-type glutenin or gliadin polypeptide such that it binds with a lower affinity to T-cells derived from a celiac patient than a corresponding non-mutated polypeptide binds to T cells derived from said celiac patient.

30. A dough comprising the flour of claim 28.

31. The dough of claim 30, characterized by at least one property selected from the group consisting of: a higher development time (DT), a lower stability time (S), a higher degree of softening (DS), a higher consistency (C) value and any combination thereof, as compared to a corresponding dough being absent of the de-epitoped glutenin or gliadin polypeptide.

32-35. (canceled)

36. A wheat being genetically modified to express the isolated glutenin or gliadin polypeptide of claim 23.

37-45. (canceled)


SEQUENCE LISTING (48 sequences)

SEQ ID NO: 1 - protein, 9 aa, Artificial sequence
  Celiac disease-associated T cell epitopes amino acid sequence
PFPQPELPY

SEQ ID NO: 2 - protein, 9 aa, Artificial sequence
  Celiac disease-associated T cell epitopes amino acid sequence
PYPQPELPY

SEQ ID NO: 3 - protein, 9 aa, Artificial sequence
  Celiac disease-associated T cell epitopes amino acid sequence
PQPELPYPQ

SEQ ID NO: 4 - protein, 9 aa, Artificial sequence
  Celiac disease-associated T cell epitopes amino acid sequence
FRPEQPYPQ

SEQ ID NO: 5 - protein, 9 aa, Artificial sequence
  Celiac disease-associated T cell epitopes amino acid sequence
PQQSFPEQQ

SEQ ID NO: 6 - protein, 9 aa, Artificial sequence
  Celiac disease-associated T cell epitopes amino acid sequence
IQPEQPAQL

SEQ ID NO: 7 - protein, 9 aa, Artificial sequence
  Celiac disease-associated T cell epitopes amino acid sequence
QQPEQPYPQ

SEQ ID NO: 8 - protein, 9 aa, Artificial sequence
  Celiac disease-associated T cell epitopes amino acid sequence
SQPEQEFPQ

SEQ ID NO: 9 - protein, 9 aa, Artificial sequence
  Celiac disease-associated T cell epitopes amino acid sequence
PQPEQEFPQ

SEQ ID NO: 10 - protein, 9 aa, Artificial sequence
  Celiac disease-associated T cell epitopes amino acid sequence
QQPEQPFPQ

SEQ ID NO: 11 - protein, 9 aa, Artificial sequence
  Celiac disease-associated T cell epitopes amino acid sequence
PQPEQPFCQ

SEQ ID NO: 12 - protein, 9 aa, Artificial sequence
  Celiac disease-associated T cell epitopes amino acid sequence
QQPFPEQPQ

SEQ ID NO: 13 - protein, 9 aa, Artificial sequence
  Celiac disease-associated T cell epitopes amino acid sequence
PFPQPEQPF

SEQ ID NO: 14 - protein, 9 aa, Artificial sequence
  Celiac disease-associated T cell epitopes amino acid sequence
PQPEQPFPW

SEQ ID NO: 15 - protein, 9 aa, Artificial sequence
  Celiac disease-associated T cell epitopes amino acid sequence
PFSEQEQPV

SEQ ID NO: 16 - protein, 9 aa, Artificial sequence
  Celiac disease-associated T cell epitopes amino acid sequence
FSQQQESPF

SEQ ID NO: 17 - protein, 9 aa, Artificial sequence
  Celiac disease-associated T cell epitopes amino acid sequence
PFPQPEQPF

SEQ ID NO: 18 - protein, 9 aa, Artificial sequence
  Celiac disease-associated T cell epitopes amino acid sequence
PQPEQPFPQ

SEQ ID NO: 19 - protein, 9 aa, Artificial sequence
  Celiac disease-associated T cell epitopes amino acid sequence
PIPEQPQPY

SEQ ID NO: 20 - protein, 9 aa, Artificial sequence
  Celiac disease-associated T cell epitopes amino acid sequence
PFPQPEQPF

SEQ ID NO: 21 - protein, 9 aa, Artificial sequence
  Celiac disease-associated T cell epitopes amino acid sequence
PQPEQPFPQ

SEQ ID NO: 22 - protein, 9 aa, Artificial sequence
  Celiac disease-associated T cell epitopes amino acid sequence
PYPEQEEPF

SEQ ID NO: 23 - protein, 9 aa, Artificial sequence
  Celiac disease-associated T cell epitopes amino acid sequence
PYPEQEQPF

SEQ ID NO: 24 - protein, 9 aa, Artificial sequence
  Celiac disease-associated T cell epitopes amino acid sequence
PFSEQEQPV

SEQ ID NO: 25 - protein, 9 aa, Artificial sequence
  Celiac disease-associated T cell epitopes amino acid sequence
EGSFQPSQE

SEQ ID NO: 26 - protein, 9 aa, Artificial sequence
  Celiac disease-associated T cell epitopes amino acid sequence
EQPQQPFPQ

SEQ ID NO: 27 - protein, 9 aa, Artificial sequence
  Celiac disease-associated T cell epitopes amino acid sequence
EQPQQPYPE

SEQ ID NO: 28 - protein, 9 aa, Artificial sequence
  Celiac disease-associated T cell epitopes amino acid sequence
QGYYPTSPQ

SEQ ID NO: 29 - protein, 9 aa, Artificial sequence
  Celiac disease-associated T cell epitopes amino acid sequence
EGSFQPSQE

SEQ ID NO: 30 - protein, 9 aa, Artificial sequence
  Celiac disease-associated T cell epitopes amino acid sequence
PQQSFPEQE

SEQ ID NO: 31 - protein, 9 aa, Artificial sequence
  Celiac disease-associated T cell epitopes amino acid sequence
QGYYPTSPQ

SEQ ID NO: 32 - protein, 290 aa, Artificial sequence
  alpha gliadin polypeptide amino acid sequence
  MISC_FEATURE (66)..(66): may be deamidated
  MISC_FEATURE (73)..(73): may be deamidated
  MISC_FEATURE (80)..(80): may be deamidated
MVRVPVPQLQPQNPSQQQPQEQVPLVQQQQFPGQQQPFPPQQPYPQPQPFPSQQPYLQLQPFPQPQLPYPQPQLPYPQPQLPYPQPQPFRPQQPYPQSQPQYSQPQQPISQQQQQQQQQQQQKQQQQQQQQILQQILQQQLIPCRDVVLQQHSIAYGSSQVLQQSTYQLVQQLCCQQLWQIPEQSRCQAIHNVVHAIILHQQQQQQQQQQQQPLSQVSFQQPQQQYPSGQGSFQPSQQNPQAQGSVQPQQLPQFEEIRNLALETLPAMCNVYIPPYCTIAPVGIFGTNYR

SEQ ID NO: 33 - protein, 15 aa, Artificial sequence
  amino acid sequence of variants that were predicted to have compromised binding to MHC
PYPHPWLRGPQPQPF

SEQ ID NO: 34 - protein, 15 aa, Artificial sequence
  amino acid sequence of variants that were predicted to have compromised binding to MHC
LQLQPFPQPQLPYPQ

SEQ ID NO: 35 - protein, 15 aa, Artificial sequence
  amino acid sequence of variants that were predicted to have compromised binding to MHC
LQLQPFPQPELPYPQ

SEQ ID NO: 36 - protein, 15 aa, Artificial sequence
  amino acid sequence of variants that were predicted to have compromised binding to MHC
LQLQPFPHPELSYSQ

SEQ ID NO: 37 - protein, 15 aa, Artificial sequence
  amino acid sequence of variants that were predicted to have compromised binding to MHC
LQLQPFDQPRLPWPQ

SEQ ID NO: 38 - protein, 15 aa, Artificial sequence
  amino acid sequence of variants that were predicted to have compromised binding to MHC
LQLQPFWQPKLPGPQ

SEQ ID NO: 39 - protein, 15 aa, Artificial sequence
  amino acid sequence of variants that were predicted to have compromised binding to MHC
QLPYPQPQLPYPQPQ

SEQ ID NO: 40 - protein, 15 aa, Artificial sequence
  amino acid sequence of variants that were predicted to have compromised binding to MHC
ELPYPQPELPYPQPQ

SEQ ID NO: 41 - protein, 15 aa, Artificial sequence
  amino acid sequence of variants that were predicted to have compromised binding to MHC
HLPYPQPELRYSQPQ

SEQ ID NO: 42 - protein, 15 aa, Artificial sequence
  amino acid sequence of variants that were predicted to have compromised binding to MHC
MLRYPQPWLPYPQPQ

SEQ ID NO: 43 - protein, 15 aa, Artificial sequence
  amino acid sequence of variants that were predicted to have compromised binding to MHC
KLPYPQGRLPGPQPQ

SEQ ID NO: 44 - protein, 15 aa, Artificial sequence
  amino acid sequence of variants that were predicted to have compromised binding to MHC
PYPQPQLPYPQPQPF

SEQ ID NO: 45 - protein, 15 aa, Artificial sequence
  amino acid sequence of variants that were predicted to have compromised binding to MHC
PYPQPELPYPQPQPF

SEQ ID NO: 46 - protein, 15 aa, Artificial sequence
  amino acid sequence of variants that were predicted to have compromised binding to MHC
PYPHPRLPYTQPQPF

SEQ ID NO: 47 - protein, 15 aa, Artificial sequence
  amino acid sequence of variants that were predicted to have compromised binding to MHC
PYSQPESPYMQPQPF

SEQ ID NO: 48 - protein, 15 aa, Artificial Sequence
  amino acid sequence of variants that were predicted to have compromised binding to MHC
PYPHPWLRGPQPQPF